(19) United States
(12) Patent Application Publication
(10) Pub. No.: US 2026/0193696 A1
(43) Pub. Date: Jul. 9, 2026

(54) METHOD FOR PROCESSING BIOMOLECULE AND DEVICE FOR PROCESSING BIOMOLECULE

(71) Applicant: Hitachi High-Tech Corporation, Tokyo (JP)

(72) Inventors: Sayaka TEZUKA, Tokyo (JP); Yoshimitsu YANAGAWA, Tokyo (JP); Ryo IMAI, Tokyo (JP); Tatsuo NAKAGAWA, Tokyo (JP)

(21) Appl. No.: 19/304,948

(22) Filed: Aug. 20, 2025

(30) Foreign Application Priority Data
Jan. 9, 2025 (JP) 2025-003166

(51) Int. Cl. C12Q 1/6844; C12Q 1/6806

(52) U.S. Cl. CPC C12Q 1/6844; C12Q 1/6806

(57) ABSTRACT

Provided is a technique for stably conveying a fluid and processing a biomolecule with a high efficiency. The processing of a biomolecule uses a flow path device in which capture and amplification of a biomolecule in an introduced sample are performed. The flow path device includes a membrane configured to capture a biomolecule contained in a sample and a space in which a liquid is accumulated before and after the membrane. A liquid containing the biomolecule is conveyed from the first path to the second path, and the biomolecule is captured on the membrane. After the capturing, the biomolecule is amplified together with the membrane using the membrane and the liquid in a space, wherein at the start of the capturing, a first space close to the first path and a second space close to the second path in the space are separated by the membrane.

### CROSS-REFERENCE TO RELATED APPLICATION

This application claims the priority of Japanese Patent Application No. 2025-003166 filed on January 9, 2025, which is incorporated herein by reference in its entirety.

### Technical Field

The present invention relates to a method for processing a biomolecule and a device for processing a biomolecule.

### Background Art

When a gene is analyzed, for example, a flow in which a sample is dissolved, a nucleic acid is purified, and amplified as pretreatment, and an amplified product is detected is adopted. This step involves a risk of contamination and complicated sample adjustment. Therefore, conventionally, it has been a general flow that a sample is sent to an environment equipped with experimental equipment such as a laboratory, and an inspector having specialized knowledge and techniques performs sample adjustment and measurement and analyzes data. However, it takes time to transport the sample, and large equipment cost and labor cost are required to maintain the experimental facility. In addition, in the case of a laboratory that introduces batch processing, it is difficult to wedge an urgent sample.

In recent years, a Sample-to-answer type analysis system that performs from introduction of a sample to measurement and acquisition of data fully automatically has been appearing in various fields. A flow path device in which a chamber, a flow path and a storage reagent are integrated is sometimes used for the Sample-to-answer type analysis system. The Sample-to-answer type analysis system using a flow path device has the following advantages. (1) Measurements can be easily performed by non-experts, (2) data can be acquired in a short period, (3) portability is high, (4) variations derived from manual operations can be reduced, and (5) storage of reagents is easy. The fields of application of the Sample-to-answer type analysis system, including potential applications, include, for example, forensic medicine, in vitro diagnosis, identification of species of animals and plants, biodefense, medicine, biotechnology, life science, defense, public health, and agriculture.

As an example of a Sample-to-answer type analysis system, as disclosed in PTL 1, PTL 2, PTL 3, NPTL 1 and NPTL 2, an analyzer configured to dissolve a human-derived sample, purify and amplify a nucleic acid, detect the nucleic acid, and perform fully automatic DNA identification is known.

PTL 4 discloses an analyzer for biological samples. PTL 4 describes that bacteria or cells trapped in a membrane installed in an amplification chamber are ground across the membrane to extract a nucleic acid, and the extracted nucleic acid is washed away with an amplification reaction liquid and amplified.

### Citation List

### Patent Literature

PTL 1: US 11649496 B

PTL 2: US 11612893 B

PTL 3: WO 2024/013952 A

PTL 4: US 10752936 B

### Non-patent Literature

NPL 1: J. Kim, et al., "A PCR reactor with an integrated alumina membrane for nucleic acid isolation," Analyst, 2010, 135, p.2408-2414

NPL 2: Y. Gu, et al., "Modular-Based Integrated Microsystem with Multiple Sample Preparation Modules for Automated Forensic DNA Typing from Reference to Challenging Samples," Analytical Chemistry, 2019, 91, 11, p.7435-7443

### Summary of Invention

### Technical Problem

The Sample-to-answer type analysis system is required to have a small analyzer size, be light, be less likely to break, and operate stably with less maintenance. In addition, a plurality of samples needs to be analyzed simultaneously or sequentially.

When gene analysis is performed by a Sample-to-answer type analysis system, in order to prevent samples from being mixed between analyses, it is desirable to make a flow path device that may directly touch a sample disposable every time measurement is performed. In order to reduce the cost of the flow path device, it is desirable to use a flow path device having a design that can be manufactured with a simple manufacturing line, or to use an inexpensive material. In particular, in order to capture and purify a sample with a purification membrane and bring the sample into amplification of a nucleic acid, a measurement mechanism of an eluate and a mechanism for mixing with a reagent are required, and thus the apparatus is complicated. As disclosed in PTL 1, PTL 3, NPL 1 and NPL 2, a membrane for capturing a nucleic acid is often installed in an amplification chamber, and an independent elution step is sometimes omitted.

PTL 1 describes that a membrane is installed in an amplification chamber, and a part of a dissolved sample is captured by the membrane and amplified. However, in PTL 1, since there is a gap through which the dissolution product can pass between the membrane and the chip, the capture efficiency is estimated to be low. In addition, application to high-sensitivity analysis at a level (for example, a forensic evidence left at a crime scene, or a case work sample) at which even a small amount of sample can be inspected with high sensitivity has not been considered.

In PTL 2, a nucleic acid contained in a dissolved sample is captured by a purification membrane, and the purified nucleic acid is liberated by an eluate, mixed with an amplification reagent, and amplified. However, the flow path structure is complicated in order to accurately meter and mix the eluate with the amplification reagent. In addition, since only a part of the eluate is mixed with the amplification reagent, loss occurs and sensitivity is estimated to be low.

According to PTL 3, it is stated that while a high-density membrane can highly efficiently capture nucleic acids contained in a dissolved sample, the pressure required for liquid feeding tends to increase, and particularly when air bubbles are included, the pressure significantly increases. If the liquid feeding pressure varies depending on the presence or absence of air bubbles, liquid feeding cannot be performed stably. In addition, it is necessary to use a complicated and expensive chip so as to withstand a high liquid feeding pressure. PTL 3 describes that a solution having a high evaporation rate or a solution having a low surface tension is continuously caused to flow after a dissolution product to reduce the pressure required for liquid feeding. However, in the pretreatment step, there is also a step in which a solution having a high evaporation rate or a solution having a low surface tension cannot be continuously conveyed. Furthermore, in a case where air bubbles are generated at an unintended timing or in a case where the membrane is clogged, the pressure required for liquid feeding increases. In addition, air bubbles are randomly generated in the flow path device. In a case where the liquid feeding parameter greatly varies depending on whether the air bubbles are caught or not, liquid feeding cannot be performed correctly. As one of the countermeasures, use of a liquid level detection sensor, etc. is conceivable, but the device becomes complicated.

In the technique described in PTL 4, it is difficult to extract a nucleic acid in a case where bacteria or cells contained in a sample are broken from the beginning, or in a case where a pretreatment is selected such that the cells or bacteria are broken before they are put into a state that they are captured by a membrane. In particular, in the case of a case work sample collected at a scene of a criminal investigation, since cells may have already been broken at the time of collection, a nucleic acid cannot be stably extracted with a high efficiency by the method described in PTL 4.

NPL 1 describes that a membrane is installed in an amplification chamber, and a nucleic acid contained in a dissolved sample is captured and amplified. NPL 1 describes that all nucleic acids captured on the membrane can be analyzed with a high sensitivity because they are all brought into amplification. Paraffin is adopted as an example of a method for fixing a membrane to a flow path. The amplification chamber is separated by a membrane into an upper part and a lower part, and the upper part and the lower part each include two inlet and outlet passes. When a dissolution product is passed through the membrane, a solution is introduced from the upper inlet path and the solution is discharged from the lower outlet path so as to cross the membrane. Subsequently, when a washing liquid and an amplification reagent are introduced into the chamber, the upper and lower inlet paths are used, and when they are discharged, the upper and lower outlet paths are used. By this method, it is possible to prevent a membrane having a large flow path resistance from hindering solution conveyance. However, since upper and lower inlet and outlet paths are required, a valve is required for each path in the case of being incorporated into a Sample-to-answer type device, and thus a flow path structure becomes complicated.

NPTL 2 describes that a membrane is installed in an amplification chamber, and a nucleic acid contained in a dissolved sample is captured and amplified. NPTL 2 describes that even a small amount of sample can be inspected with a high sensitivity. However, it does not meet the level of analysis sensitivity implemented by forensic laboratories. Further, the stability of solution conveyance has not been studied.

Sample-to-answer type devices require rapidity of analysis. Therefore, the time required for solution conveyance and nucleic acid amplification needs to be short. In summary, a flow path device is required to have a simple flow path structure, to be able to stably convey a solution, and to be able to capture a nucleic acid with a high efficiency. Then, the flow path device is required to be able to analyze a nucleic acid with a high sensitivity and in a short period.

Therefore, the present disclosure provides a technique for stably conveying a fluid and processing a biomolecule with a high efficiency.

### Solution to Problem

In order to solve the above-mentioned problem, the present disclosure is a method for processing a biomolecule using a flow path device configured to capture a biomolecule in a sample to be introduced, the flow path device including: a membrane configured to capture the biomolecule contained in the sample; a space in which a liquid is accumulated before and after the membrane; and a first path and a second path connected to the space, the method for processing a biomolecule including conveying a first liquid containing the biomolecule from the first path to the second path, and capturing the biomolecule on the membrane, and after the capturing, amplifying the biomolecule together with the membrane using the membrane and the liquid in the space, wherein during the capturing, a first space close to the first path and a second space close to the second path in the space are separated by the membrane, and newly forming a third path through which a fluid can move between the first space and the second space during from the end of the capturing to the end of the amplification.

Further features related to the present disclosure will become apparent from the description of the present specification and the accompanying drawings. In addition, the aspects of the present disclosure are achieved and realized by elements, combinations of various elements, the following detailed description, and aspects of the appended claims. The description of the present specification is merely exemplary, and does not limit the scope of claims or application examples of the present disclosure in any sense.

### Advantageous Effects of Invention

According to the technology of the present disclosure, it is possible to stably convey a fluid and process a biomolecule with a high efficiency. Problems, configurations and effects other than those described above will be clarified by the following description of embodiments.

### BRIEF DESCRIPTION OF DRAWINGS

FIG. 1 is a block diagram showing a schematic configuration of the analysis system of the present disclosure.

FIG. 2 is a schematic configuration diagram illustrating an example of the flow path device of the analysis system.

FIG. 3 is a flowchart illustrating an example of an operation procedure of the analysis system.

FIG. 4A is a cross-sectional view showing an example of a method for installing a membrane.

FIG. 4B is a cross-sectional view showing an example of a method for installing a membrane.

FIG. 4C is a cross-sectional view showing an example of a method for installing a membrane.

FIG. 4D is a cross-sectional view showing an example of a method for installing a membrane.

FIG. 4E is a cross-sectional view showing an example of a method for installing a membrane.

FIG. 4F is a cross-sectional view showing an example of a method for installing a membrane.

FIG. 4G is a cross-sectional view showing an example of a method for installing a membrane.

FIG. 5 is a top view illustrating a detailed configuration example of the flow path device.

FIG. 6 is a flowchart illustrating an operation procedure in the flow path device.

FIG. 7A is a cross-sectional view showing an example of a method for amplification together with a membrane in a nucleic acid capturing/amplification chamber.

FIG. 7B is a cross-sectional view showing an example of a method for amplification together with a membrane in a nucleic acid capturing/amplification chamber.

FIG. 7C is a cross-sectional view showing an example of a method for amplification together with a membrane in a nucleic acid capturing/amplification chamber.

FIG. 7D is a cross-sectional view showing an example of a method for amplification together with a membrane in a nucleic acid capturing/amplification chamber.

FIG. 8A is an example of an STR-CE profile.

FIG. 8B is an example of an STR-CE profile.

FIG. 9 is a cross-sectional view for explaining a problem in the case of conveying an amplification reagent to a nucleic acid capturing/amplification chamber.

FIG. 10A is a top view for explaining a problem in the case of conveying an amplified product from the nucleic acid capturing/amplification chamber.

FIG. 10B is a top view for explaining a problem in the case of conveying an amplified product from the nucleic acid capturing/amplification chamber.

FIG. 11A is a view showing a method for forming a third path in the nucleic acid capturing/amplification chamber of the present disclosure.

FIG. 11B is a view showing a method for forming a third path in the nucleic acid capturing/amplification chamber of the present disclosure.

FIG. 12 is a view for explaining an effect of eliminating clogging by the third path.

FIG. 13 is a view for explaining an effect of preventing bias of components by the third path.

FIG. 14A is a view illustrating an example of a method for forming the third path.

FIG. 14B is a view illustrating an example of a method for forming the third path.

FIG. 14C is a view illustrating an example of a method for forming the third path.

FIG. 14D is a view illustrating an example of a method for forming the third path.

FIG. 14E is a view illustrating an example of a method for forming the third path.

FIG. 14F is a view illustrating an example of a method for forming the third path.

FIG. 14G is a view illustrating an example of a method for forming the third path.

FIG. 14H is a view illustrating an example of a method for forming the third path.

FIG. 14I is a view illustrating an example of a method for forming the third path.

### DESCRIPTION OF EMBODIMENTS

### Description of Terms

In the present specification, the “flow path device” refers to a flow path device including a flow path substrate (chip) on which a groove mainly serving as a flow path is dug and a film bonded to the flow path so as to cover the flow path. However, flow path devices having different configurations that provide similar functions may also be used.

In the present specification, “joined” refers to a state in which different members are joined mainly by an adhesive, heat welding, screwing, fitting, etc. However, even a single member integrally molded from the beginning is encompassed in the “joined” state as long as it has portions that play different roles inside.

The “biomolecule” refers to nucleic acids, proteins, lipids, polysaccharides, amino acids, lipids, sugars, nucleobases, physiologically active substances, derivatives thereof, and complexes thereof.

In the present specification, the target of the biomolecule to be captured, purified and reacted is mainly a nucleic acid (particularly DNA), but other biomolecules may be targeted. In particular, the present method can be applied to an operation in a flow path device including a step of capturing a biomolecule of interest on a membrane and purifying the biomolecule using a washing liquid, etc., or reacting the captured biomolecule.

In the present disclosure, the reaction mainly refers to an amplification reaction, but may be other reactions. The reaction of the present disclosure is not limited to the amplification reaction, and the captured nucleic acid may be used in other nucleic acid engineering reactions that do not involve amplification, such as Ligation, Hybridization, crosslink, single base elongation reaction, Restriction enzymedigestion, and cleavage by Crisper/Cas. Alternatively, the protein may be captured by a membrane and detected with an antibody.

A probe DNA may be bound to a molecule (protein, etc.) other than the nucleic acid captured on the membrane, and said DNA may be amplified to be used for a method for detecting a target molecule.

When a nucleic acid is amplified, for example, a polymerase chain reaction (PCR) method, a Loop-Mediated Isothermal Amplification (LAMP) method, a Rolling circle amplification (RCA) method, a Reverse transcription-PCR (RT-PCR) method, a Transcription Reverse-transcription Concerted reaction (TRC) method, or Nucleic Acid Sequence-Based Amplification (NASBA) can be used. In the present specification, a case where a PCR method is mainly used will be described, but other amplification methods may also be used.

In the present specification, the term “STR-CE” refers to a series of flows of adjusting an amplification reaction solution using short tandem repeats (STR) as an amplification target, performing an amplification reaction (STR-PCR), measuring with a capillary electrophoresis device (CE), and analyzing an obtained electropherogram.

In the present specification, STR-PCR is mainly described, but the application of the nucleic acid capturing/amplifying device of the present disclosure is not limited thereto. Other examples of amplification targets may include genetic mutation analysis or quantification, cell line authentication, determination of genome editing efficiency, amplification fragment length polymorphism (AFLP), simple sequence repeat (SSR), single nucleotide polymorphism (SNP) genotyping, and macrosatellite markers. Markers of infectious diseases or various diseases, etc. may also be amplification targets.

In the present specification, the membrane is at least one selected from the group consisting of cellulose membranes, chitosan membranes, glass fiber membranes, plastics, ceramic sintered bodies, filter paper, nonwoven fabrics, cotton, threads, and aggregates of particles capable of capturing nucleic acids.

In the present specification, when nucleic acid is “captured” on a membrane, hydrophobic interaction, van der Waals force, or ionic interaction may act between the nucleic acid and the membrane. The nucleic acid may be encased or captured in another particle, and such a particle may be captured by the membrane.

In the present specification, the “nucleic acid capturing/amplification chamber” refers to a chamber including a membrane and capable of storing a solution. The nucleic acid capturing/amplification chamber may be configured to be in contact with a heat source or to be applied with heat in an analyzer. When the nucleic acid capturing/amplification chamber is heated during the amplification reaction, both ends can be closed with valves to prevent the solution from overflowing or evaporating from the chamber.

In the present specification, “amplification together with a membrane” refers to performing an amplification reaction by bringing a membrane capturing a nucleic acid into contact with an amplification reagent. It is sufficient that the time during which the membrane is in contact with the reagent before the start of the amplification reaction is secured for 1 second or more, and the membrane and the reagent are not necessarily in contact with each other during the amplification reaction.

In the present specification, the “capillary electrophoresis analysis (CE analysis)” refers to a series of flows in which an electrophoresis sample is prepared, capillary electrophoresis measurement is performed, an electropherogram is acquired, and DNA identification or fragment analysis is performed. However, a part of the step may not be included in the range indicated by “CE analysis”.

In the present specification, a nucleic acid obtained by an amplification reaction is referred to as an “amplified product”. In the present specification, a mixture of an amplification reaction liquid and an electrophoresis reagent is referred to as an “electrophoresis sample”. When the electrophoretic sample is prepared, a heating step may be performed because the nucleic acid is easily denatured into a single strand when heated to 90°C or higher, and more accurate CE analysis can be performed. The “electrophoresis sample” may be a sample before denaturation by heating or a sample after denaturation.

### Configuration Example of Analysis System

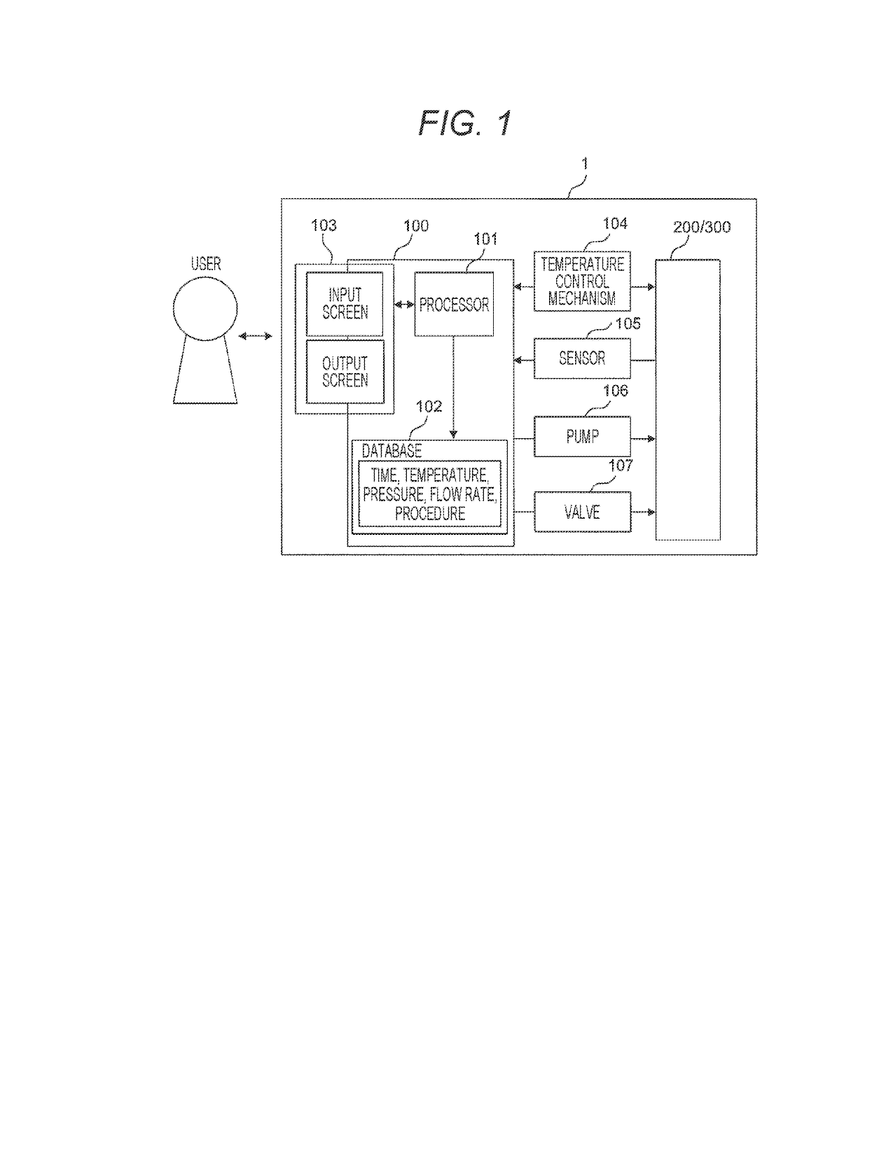

FIG. 1 is a block diagram showing a schematic configuration of an analysis system 1 of the present disclosure. The analysis system 1 is a device (a Sample-to-answer type device) configured to analyze a biomolecule (for example, a nucleic acid) by pretreating a sample derived from a living body. The analysis system 1 includes a computer 100, a flow path device 200, a detection unit 300, a temperature control mechanism 104, a sensor 105, a pump 106, and a valve 107.

A sample is introduced into the flow path device 200. The flow path device 200 pretreats the sample, and the pretreated sample is sent to the detection unit 300. As will be described in detail later, the flow path device 200 has a flow path through which a sample flows, a reagent tank that holds a reagent necessary for pretreatment, and a chamber that reacts the sample with the reagent. The detection unit 300 includes, for example, one or more capillaries installed in a capillary electrophoresis device.

The flow path device 200 may be disposable. By being disposable, contamination between samples can be prevented. The detection unit 300 may be disposable. By being disposable, contamination between samples can be prevented. While the flow path device 200 is disposable, the detection unit 300 may be usable multiple times. Since the detection unit 300 is required to be manufactured precisely and has a high unit price, it is possible to reduce the cost by making it reusable.

The flow path device 200 and the detection unit 300 may be integrated together. The integrated structure facilitates storage, maintenance and transportation. With the integrated structure, the connection portion between the flow path device 200 and the detection unit 300 becomes simple, and the frequency of failure or error can be reduced.

The temperature control mechanism 104 regulates the temperature of the flow path device 200. The temperature control mechanism 104 may have a heater that can contact the flow path device 200. The temperature control mechanism 104 may include a heat source such as a Peltier or an electric heating wire. The temperature control mechanism 104 may control the temperature by blowing air to the flow path device 200, or may control the temperature in a non-contact manner by a laser or an electromagnetic wave. The output of the temperature control mechanism 104 can be controlled by the computer 100. The temperature control mechanism 104 can be used for sample dissolution, purification, and nucleic acid amplification reaction, and may include a plurality of temperature control mechanisms independent for each application. The sensor 105 is, for example, a thermocouple or a photodetector. The sensor 105 monitors the temperature of flow path device 200 and feeds back the monitored temperature to the computer 100. As a result, the temperature of the flow path device 200 can be precisely controlled.

A pump 106 and a valve 107 are solution conveying mechanisms in the analysis system 1. As the pump 106, for example, a diaphragm pump, a syringe pump, or an electrochemical pump can be used. As a conveying mechanism in place of the pump 106 or in combination with the pump 106, for example, Passive conveyance using surface tension, centrifugal force, and a combination thereof can be used.

As an example of the valve 107, a valve that directly/indirectly transmits motor power to deform a film, or a valve that deforms by air pressure can be used. Alternatively, the valve 107 may be opened and closed by being deformed by heat, or a magnetic force may be used. The pump 106 and the valve 107 can be controlled by the computer 100.

The computer 100 includes a memory (not illustrated) configured to store a program instruction, a processor 101 that is configured to execute the program instruction, a database 102 and a user interface 103. The processor 101 executes a program instruction to perform a function of receiving and analyzing raw data, optical data and electropherogram data from the detection unit 300, and a solution conveyance control function such as the pump 106 and the valve 107. The processor 101 is connected to a network and can upload data to the database 102, collate the data in the database 102 and access the data from the database 102.

Various parameters related to the analysis protocol may be stored in the database 102 in advance. Based on the parameters recorded in the database 102, the processor 101 controls opening and closing of valves of the flow path device 200 and the detection unit 300, the connection units thereof, etc., the temperature control mechanism 104, and the applied pressure/flow rate. The parameters include temperature, time, pressure, flow rate, and storage parameters. The database 102 may store a function for setting a parameter based on an actual measurement value.

The user interface 103 includes an input screen and an output screen. The user interface 103 receives, for example, various parameters such as time, temperature, pressure, flow rate, procedure, divided liquid amount and the number of amplification cycles of each step, sample information, cartridge information, analysis protocol, etc. from a user. The processor 101 stores the information input from the user interface 103 in the database 102.

The flow path device 200 that is consumed for each measurement may have a tag therein. The computer 100 may be configured to set an appropriate analysis protocol by reading information of the tag of the flow path device 200.

The computer 100 may be configured to receive a sample and perform dissolution, purification, amplification, detection and analysis in a fully automated manner. A part of the dissolution to purification to amplification to detection and analysis may be configured to be performed fully automatically. The analysis time per analysis using the flow path device 200 is typically within 2 days, and in some cases within 12 hours, or within 2 hours.

### Outline of Flow Path Device

In the present disclosure, the flow path device 200 is a consumable component that includes a reagent, a chamber, and a flow path therein, and can be disposable or used a plurality of times. The flow path device 200 may internally include a pump that is a power source for conveying a fluid. Some or all of the reagents necessary for the reaction may be present in the flow path device 200. A part of the chamber may have a temperature control function, a function of capturing molecules, a detection function or a voltage application function.

The material used for the flow path device 200 is not particularly limited as long as it is a material generally used in the art. For example, polypropylene, polyethylene, cyclic olefin polymers (COPs), cyclic olefin copolymers (COCs), polycarbonates, polyethylene terephthalates, polyurethanes, etc. can be used as materials having a small amount of adsorbed nucleic acid. Furthermore, the adsorption amount can also be suppressed by modifying the surface so as to be negatively charged. Examples of other materials include metals such as gold, silver, copper, aluminum, tungsten, molybdenum, chromium, platinum, titanium and nickel; alloys such as stainless steel, hastelloy, inconel, monel and duralumin; silicon; glass materials such as glass, quartz glass, fused quartz, synthetic quartz, alumina, sapphire, ceramics, forsterite and photosensitive glass; plastics such as polyester resins, polystyrene, polyethylene resins, ABS resins (acrylonitrile-butadiene-styrene resins), dimethylpolysiloxanes (PDMS), polyamides, acrylic resins, fluororesins, polycarbonate resins, polyurethane resins, methylpentene resins, phenol resins, melamine resins, epoxy resins and vinyl chloride resins; agarose, dextran, cellulose, polyvinyl alcohol, nitrocellulose, chitin, chitosan, or any combinations thereof.

In one embodiment, the flow path device 200 has a flow path therein and is made entirely of a deformable material. In one embodiment, the flow path device 200 is made by bonding a lid of a film to a flow path substrate having holes or grooves dug therein. In one embodiment, in the flow path device 200, the portion corresponding to the lid and the portion corresponding to the flow path are made of the same material.

In one embodiment, the parts constituting the flow path device 200 are manufactured by, for example, injection molding, a 3D printer, cutting, blow molding, extrusion molding, press molding, etc. In the case of injection molding, there is an advantage that the manufacturing cost of the flow path device 200 can be reduced in mass production. When the flow path device 200 is made of an inexpensive material, a plastically deformable material such as polypropylene, polyethylene, or PET can be used.

The size of the flow path device 200 is 50 cm or less on one side. In particular, the flow path device 200 capable of analyzing one sample has a side of 20 cm or less. When the apparatus is housed compactly, for example, the long side of the flow path device 200 can be within 15 cm, the width can be within 10 cm, and the thickness can be within 1 cm.

### Chamber/reagent tank

A chamber or reagent tank refers to a space capable of storing a liquid or solid and allowing the solution to react, wait, heat or change. The chamber may have a larger diameter than the flow path, but may not be visually distinguishable from the flow path. The chamber may have a membrane or a microstructure inside, may be formed with a composition different from that of the flow path, may have a different surface treatment, or may have a different hydrophilicity. A heater or a laser light source may be provided outside the flow path device 200. The reagent may be stored in the chamber, and amplification of the nucleic acid, dissolution of the sample, purification, etc. may be performed in the chamber. The volume of the chamber can be, for example, 0.01 μL to 50 mL.

The flow path device 200 may have two or more chambers, and each chamber is connected by a flow path. A valve may be provided between the chambers to prevent the liquid from transferring at an unintended timing.

The reagent may be stored in the flow path device 200, and the reagent may be supplied from the outside of the flow path device or from the inside of the analysis system.

As one embodiment, one or more kinds of reagents are stored in one or more reagent tanks in the flow path device 200. The reagent contains at least one of dissolution liquids, washing liquids, amplification reagents which may contain polymerases, primers, surfactants, etc., formamide, pure water, nucleic acid fragments, oils, etc. When these reagents are mixed at an unintended timing, performance may be deteriorated and other unexpected results may be caused. Therefore, the reagent can be separated by a partition mechanism including a valve, a film, air, a flow path so narrow as to prevent spontaneous mixing, or a combination thereof until immediately before use. In addition, by isolating the reagent from the outside air, long-term storage and portability of the flow path device 200 are realized. When the same reagent is released in a plurality of steps, the reagent may be stored in a plurality of reagent tanks. Similarly, when the reagent is stored outside the flow path device 200, the reagent is stored in a state of being isolated from outside air, and is separated from other components of the analysis system 1 by a valve, a film, air, etc. Known reagent storage techniques include, for example, blister reagent tanks and reagent tanks mounted on the devices of PTLS 1 and 2, and forms similar to these may be incorporated in the present disclosure. In PTL 2, a reagent tank is sealed with a film that is easily broken when pressure is applied, and the internal reagent is released into a flow path by applying pressure. In the case of such a reagent tank, the reagent can be opened and released only by the opening/closing operation of the valve, so that the device can be manufactured in a small size.

### Purification Chemistry

When nucleic acid is captured in the flow path device 200, purification chemistry used in an environment with experimental facilities may be used. Examples of a method for capturing a nucleic acid with a membrane include a Boom method, ion exchange, ChargeSwitch, and a chitosan coat membrane (NPL 2). In the Boom method, a sample is dissolved in a dissolution liquid containing a chaotropic salt and captured with a purification carrier such as a silica membrane or silica beads. After the capturing, the purification carrier is washed with a washing liquid containing ethanol or isopropanol. Then, impurities such as chaotropic salt inhibiting amplification and hematin or humic acid contained in the sample are washed away. The captured nucleic acid is released in pure water or a solution having a low salt concentration. The higher the temperature of the liquid during release and the longer the contact time, the more nucleic acids are released.

In the purification chemistry other than the Boom method, the nucleic acid in the dissolution product can be similarly captured and washed with a washing liquid. The washing step may not be performed. Hereinafter, purification by the Boom method will be described in the present disclosure, but other purification methods may be used.

### Dissolution Product

When conveying the sample to the membrane chamber, the sample needs to be in a form capable of flowing through the flow path. Therefore, in a case where the state of the sample is solid (for example, a swab sample), the solid sample can be dissolved or suspended in a dissolution buffer to obtain a fluid dissolution product. The sample need not be completely dissolved, and sites that exhibit solid or high viscosity after dissolution may be retained in the dissolution chamber. In addition, when the sample is a gas sample (for example, air, exhalation, etc.), a liquid sample can be obtained by suspending cells contained in the gas sample in a solvent. The preparation method for making a sample into a dissolution product is customary in the art and can be readily understood by anyone skilled in the art. For example, the dissolution buffer can comprise a chlorinated material such as calcium hypochlorite. As another example, the substances in the dissolution buffer can include enzymes such as nucleases and proteases. If necessary, a substance that allows a biomolecule to be easily liberated, such as Chaotrope, a surfactant, or potassium hydroxide (KOH), or a substance that allows a nucleic acid to be easily bound to the purification membrane may be added to the dissolution buffer. If necessary, the mixture may be subjected to treatments such as heating and stirring.

The substance that allows the nucleic acid to easily bind to the membrane is stored in another reagent tank as a binding liquid, and may be introduced at another timing. The binding liquid may contain chaotropic, and may contain a high concentration of salt. Furthermore, the binding liquid may contain a low pH solution such as hydrochloric acid.

In addition, the dissolution may be promoted by performing bubbling during the dissolution. As a secondary effect, DNA can be more efficiently released by fragmenting DNA by bubbling.

In the present embodiment, the “dissolution product” means a substance obtained by converting a sample derived from a living body into a liquid having a viscosity of 100,000 mPa·s or less using a dissolution buffer. Optionally, the dissolution product may have a viscosity of 10,000 mPa·s or less. Optionally, the dissolution product may have a viscosity of 1000 mPa·s or less.

### Washing Liquid

In the present disclosure, the “washing liquid” means a liquid that is used to wash away substances adhered to the purification membrane and unnecessary for subsequent steps. The washing liquid may not be able to wash away all unnecessary substances, and may wash away some or all necessary substances. In the Boom method, ethanol or IPA is often used as the washing liquid. As the concentration of ethanol, a concentration adjusted in a range of 50% to 100% is used. In addition, an aqueous solution having a pH of 7 or more may be used as the washing liquid. In addition, washing may not be performed.

### Amplification Reagent, Electrophoresis Reagent

In a typical STR-PCR analysis, two or more loci are detected. More typically, 5 or more, 10 or more, 15 or more, 20 or more, or 25 or more loci are included. As the STR-CE, for example, those sold as kits such as GlobalFiler (trademark) and PowerPlex (registered trademark) can be used. In addition, the gene locus to be detected may include a gene locus for forensic medicine or for DNA determination in each country such as CODIS, or a gene locus specified in various gene databases.

The electrophoresis reagent may include deionized formamide, size standard, and pure water. Formamide or pure water may be included to reduce the ionic strength of the electrophoretic sample or denature the nucleic acid. As the electrophoresis reagent, not only formamide or pure water but also a low-conductivity solution may be used. The low conductivity solution can have a conductivity of 10 mS/cm or less, optionally 1 mS/cm or less, 100 μS/cm or less, or 10 μS/cm or less. As the conductivity of the solution used for the electrophoresis reagent is lower, the amount of nucleic acid injected into the capillary electrophoresis device tends to increase. The size standard may be mixed to associate the detected peak with the nucleic acid length, or may be mixed to estimate the amount of nucleic acid contained in the electrophoresis sample from the detected peak.

### Amplification Reaction

An amplification reagent may be provided in the flow path device 200. As the amplification reagent, a solution containing a polymerase and a solution containing a primer may be separately provided. The amplification reagent may be a dry reagent. The sample itself, such as a swab, may be subjected to amplification. A nucleic acid purified by silica purification, Chelex (registered trademark), phenol chloroform, etc. may be mixed with an amplification reagent. The amplification reagent may be mixed with a membrane (such as a silica membrane) in which the nucleic acid is trapped.

The amplification reagent may include an internal positive control (IPC) to be amplified together with the sample nucleic acid and a set of primers for amplifying the IPC. The primer for IPC may be labeled with a dye and may be detectable by a capillary electrophoresis device. Amplicons derived from IPCs can be utilized for analysis. The nucleic acid amount of the sample may be estimated by using the intensity ratio between the IPC and the peak derived from the sample, and the amplification efficiency correction coefficient or the fluorescence intensity correction coefficient. Whether the amplification reaction is normally performed or inhibited may be estimated by confirming the intensity of the IPC.

The liquid amount of the amplification reaction solution is, for example, 1 μL to 200 μL, and depending on the case, 10 μL to 50 μL. When the liquid amount is small, there are advantages that accurate temperature control is possible, high-speed amplification is possible, and reagent cost is low. On the other hand, when the liquid amount is large, not only more inhibitor resistance can be obtained, but also the influence can be alleviated even if molecules are adsorbed to the surface of the flow path substrate or the membrane.

The PCR reaction may consist of an initial denaturation step, an annealing step, an extension step, a denaturation step, a final extension step, and may be devoid of some steps. The initial denaturation step can be heated at 90°C to 99°C for 1 s to 2 min at the start of amplification to start the reaction of amplification. In the annealing step, heating is performed at 50°C to 80°C for 1 s to 2 min to bind the primer to a template nucleic acid. The elongation step is heated at 50°C to 80°C for 1 s to 2 min, and the temperature is raised to a temperature at which the nucleic acid polymerase works well to perform the elongation reaction of the nucleic acid. The denaturation step is heated at 80°C to 99°C for 1 s to 2 min. The final extension step is heated at 50°C to 80°C for 1min to 60min. By providing the final extension step, the lengths of the amplified products can be made uniform. The annealing step, the extension step and the denaturation step are repeated 10 to 40 times. The annealing step and the extension step may be performed at the same temperature.

### Sample Type

The sample provided to the analysis system 1 of the present disclosure is not particularly limited as long as it is a sample derived from a living body. The organism from which the sample is derived is not particularly limited, and samples derived from any organism such as vertebrates (for example, mammals, birds, reptiles, fish, amphibians, etc.), invertebrates (for example, insects, nematodes, crustaceans, etc.), plants, protists, fungi, bacteria, archaea and viruses can be used. The sample can be collected using a swab, filter paper, cloth, etc. as a carrier. The carrier may be introduced into the analysis system 1.

The forensic sample includes buccal swabs, bones, muscle tissues, human organs, samples containing a very small amount of DNA called Touch samples, blood marks, skin pieces, hairs, body fluids, and residues assumed to be attached thereto. Many forensic samples contain unknown amounts of DNA, with concentrations between 0.001 ng and 1000 μg of DNA and more frequently between 0.01 ng and 10 μg of DNA. The forensic sample may also include only nucleic acids of a single person, or may include nucleic acids of multiple persons, and may include degraded DNA.

In order to increase the success rate of DNA identification, it is necessary to minimize loss due to surface adsorption of nucleic acid in the flow path device 200.

### Solution Conveyance Control

As solution conveying means of the flow path device 200, a pump and a valve can be used. As the pump, for example, a syringe pump, a diaphragm pump, an electrochemical pump, Passive conveying using surface tension, a centrifugal force, and a combination thereof can be used. Valves are used to specify the delivery path of the solution as well as to switch the path in which air pressure is applied. As the valve, for example, a diaphragm valve operated by air pressure, a mechanical valve, or a valve using surface tension can be used. The conveyable flow path may be switched by a difference in pressure required for conveyance.

### Detection Method

After the amplification, detection by a capillary electrophoresis device (CE) is performed. In the capillary electrophoresis device, a method of injecting an amplified product into a capillary filled with a polymer by voltage injection may be used. Further, when a high voltage is applied across the capillary, the nucleic acid fragments that fluoresce are separated by size and detected with a laser/camera system. Reference is made herein primarily to CE analysis. Instead of the CE analysis, Massively parallel sequencing (MPS), pyrosequencing, Sanger sequencing, nanopore sequencing, chromatography, electrical measurement, spectroscopy, NMR, RFLP (Restriction Fragment Length Polymorphisms), microarray, etc. may be used.

As a known example of the nucleic acid analysis and conveyance procedure, it is possible to refer to PTLS 1 and 2, home page <URL: https://www.qiagen.com/ja-us/products/human-id-and-forensics/investigator-solutions/qiaamp-dna-investigator-kit> of QIAamp (registered trademark) DNA Investigator Kit of QIAGEN N.V.

### Outline of Flow Path Device

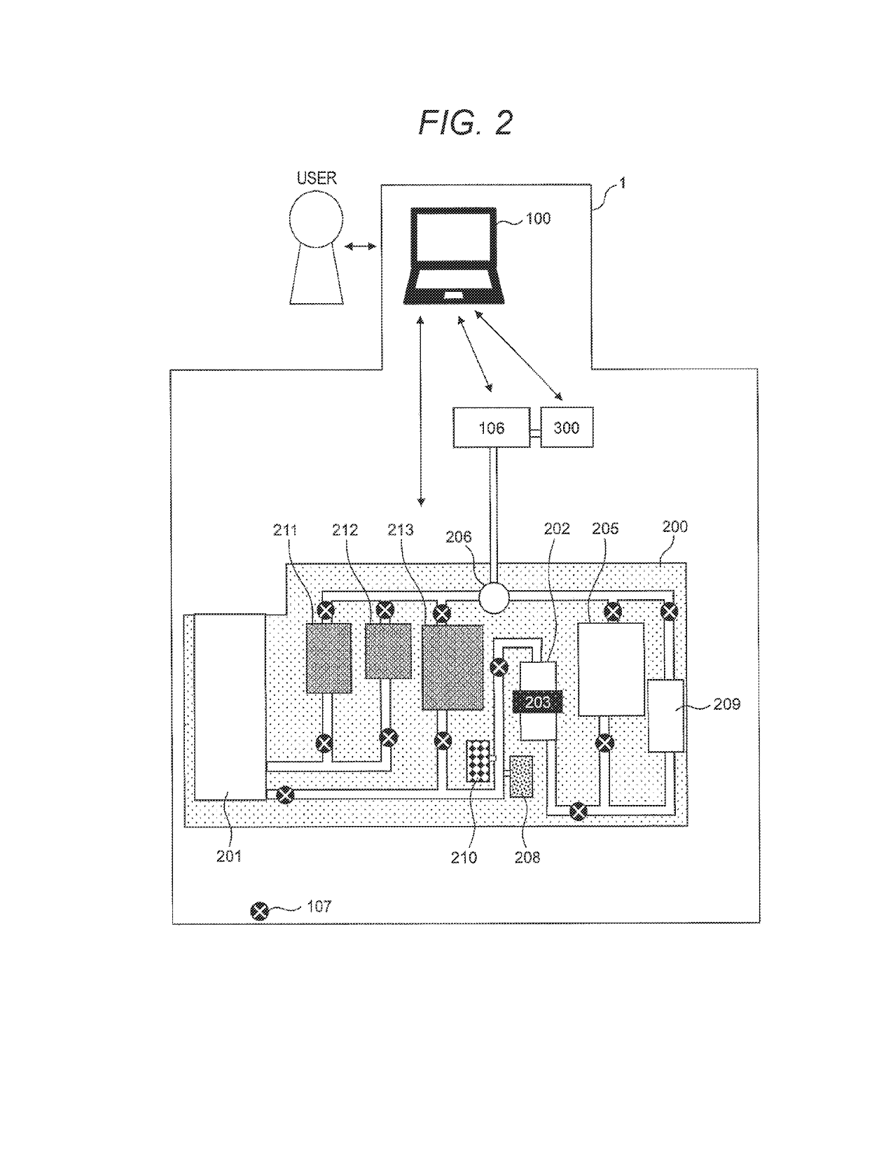

FIG. 2 is a schematic configuration diagram illustrating an example of the flow path device 200 of the analysis system 1. The flow path device 200 includes a dissolution chamber 201, a nucleic acid capturing/amplification chamber 202, a waste liquid chamber 205, an external connection port 206, a mixing chamber 209, and reagent tanks 208, 210, 211, 212 and 213. The chambers 201, 202, and 205, and the reagent tanks 208, 210, 211, 212 and 213 are provided in the middle of the flow path or to join the flow path. The flow path is provided with a plurality of valves.

The collected sample is introduced into the dissolution chamber 201 and dissolved. The nucleic acid capturing/amplification chamber 202 stores a membrane 203. The external connection port 206 is fluidly connected to the outside of the flow path device 200. The solution is conveyed through the external connection port 206, and a reagent, an amplified product, etc. can be exchanged with the outside of the flow path device 200. When the solution is conveyed in the analysis system 1, the solution can be fed using a conveyance mechanism including a pump 106 and a valve 107. All of the pumps may be provided outside the flow path device 200, or some of the pumps may be provided inside the flow path device 200. In addition, the reagent tank 208 stores reagents (polymerases, primers, dNTPs, buffers, etc.) necessary for the amplification reaction. The reagent tank 210 stores an electrophoresis reagent. The amplification reagent may be stored in a liquid state or may be stored in a solid state. In the case of a solid state, a solution for dissolution may be stored separately, and when the solid amplification reagent is dissolved, the solution may be dissolved in the nucleic acid capturing/amplification chamber 202, or the solution may be introduced into the nucleic acid capturing/amplification chamber 202 in a state of being dissolved in advance at another place. Furthermore, as disclosed in U.S. Patent No. 9409166, a reagent tank may be provided in the nucleic acid capturing/amplification chamber 202. The reagent tank may be divided into two or more regions, in which case the contents may be the same or different. The reagent tanks 211, 212 and 213 store reagents necessary for a pretreatment of the sample. The reagent tank 211 houses a dissolution liquid. The reagent tank 212 houses a binding liquid. The reagent tank 213 houses a washing liquid.

### Operation of Analysis System

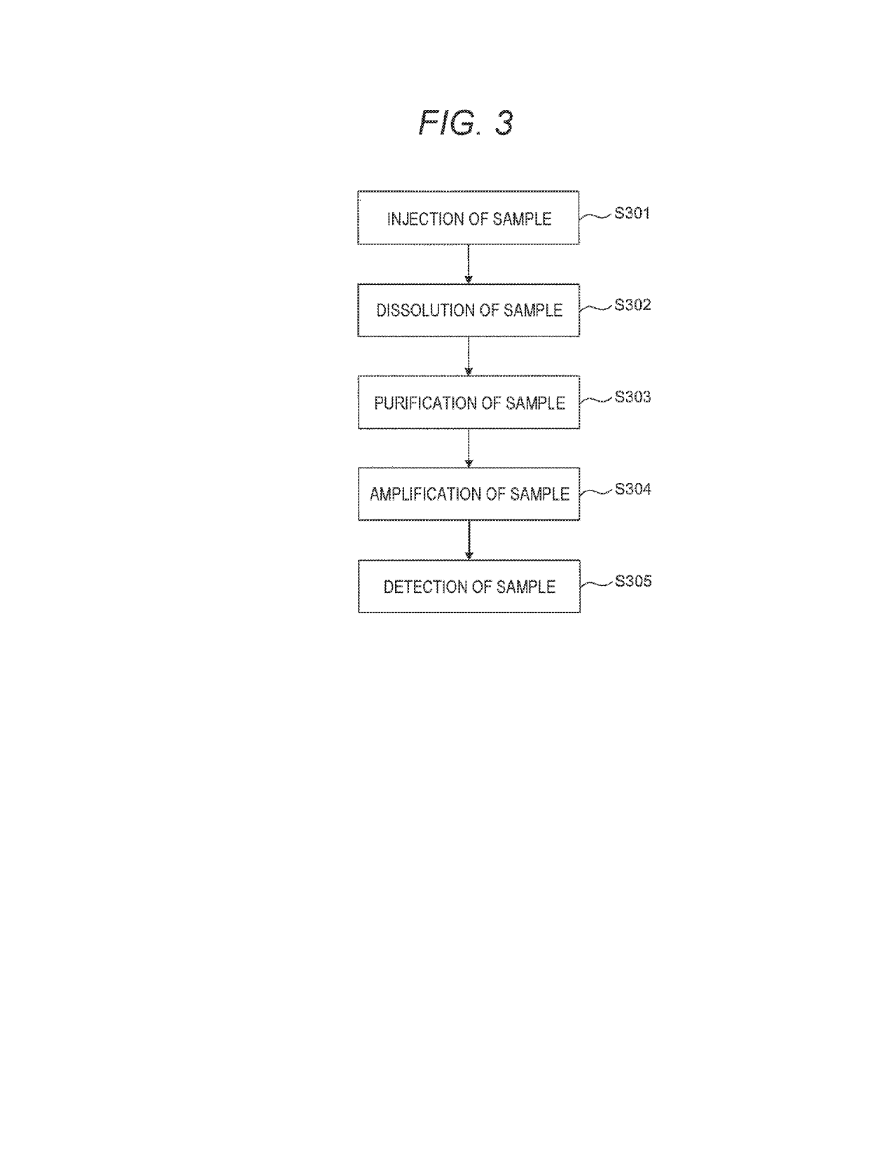

FIG. 3 is a flowchart illustrating an example of an operation procedure of the analysis system 1. In step S301, a sample is introduced into the dissolution chamber 201. Before and after that, the computer 100 controls the valve and the pump to convey the dissolution liquid from the reagent tank 211 to the dissolution chamber 201. In step S302, dissolution starts. After the end of the dissolution, the binding liquid is released from the reagent tank 212.

In step S303, the computer 100 controls valves and pumps to convey the dissolution product from the dissolution chamber 201 to the nucleic acid capturing/amplification chamber 202. Thus, the nucleic acid is captured by the membrane 203. Then, the washing liquid is discharged from the reagent tank 213, and the sample is purified. After the purification, a step of drying the washing liquid, etc. may be performed.

In step S304, the computer 100 controls the valve and the pump to release the amplification reagent from the reagent tank 208 and convey the same to the nucleic acid capturing/amplification chamber 202. As a result, the nucleic acid captured by the membrane 203 is amplified.

In step S305, the computer 100 controls the valve and the pump to mix the amplified nucleic acid with the electrophoresis reagent stored in the reagent tank 210. These mixed liquids are conveyed to the detection unit 300 (CE unit), and measurement is performed. After mixing the electrophoresis reagent and the amplification reaction liquid, a step of heating at 80 to 100°C and rapidly cooling the mixture to 0°C to 10°C may be provided before CE analysis. By providing this step, the nucleic acid is more completely single-stranded, enabling highly accurate CE analysis.

### Membrane

The nucleic acid capturing/amplification chamber 202 is provided with a membrane 203 (purification membrane), and the nucleic acid in the dissolution product is captured by the membrane 203 and a washing liquid is caused to flow to remove impurities. The purified nucleic acid may be amplified while remaining attached to the membrane 203 or may be amplified off of the membrane 203.

Examples of the type of the membrane 203 include a silica membrane containing silica as a main component. Other examples of the membrane 203 can include a solid substrate comprising cellulose, which is capable of adsorbing nucleic acids, as a main component, carboxylated particles, and an ion exchange resin. In particular, a membrane having a hydroxyl group or a silica group on the surface can be used. The membrane 203 may be any membrane as long as it can hold particles of 100 μm or more. The thickness can be 1 μm or more. More preferably, the thickness can be set to 0.1 mm to 5 mm. Furthermore, since the finer the size, the more efficiently the nucleic acid can be recovered, a membrane capable of holding particles of 10 μm or more, 1 μm or more, or 0.1 μm or more can be used. In the case of using a coarse membrane, it is possible to maintain highly efficient trapping by reducing the conveyance speed of the liquid.

If the volume of the membrane 203 is too small, the amount of biomolecule that can be adsorbed is reduced. On the other hand, when the volume of the membrane 203 is too large, there is a concern that the probability of occurrence of unintended molecular adsorption in purification or a subsequent step increases, or the conveyance efficiency of the solution deteriorates. In each embodiment described later, a membrane having an area of 12.5 mm² is used, but for example, a membrane having an area of 1 mm² to 314 mm² can also be used, and the size is not limited.

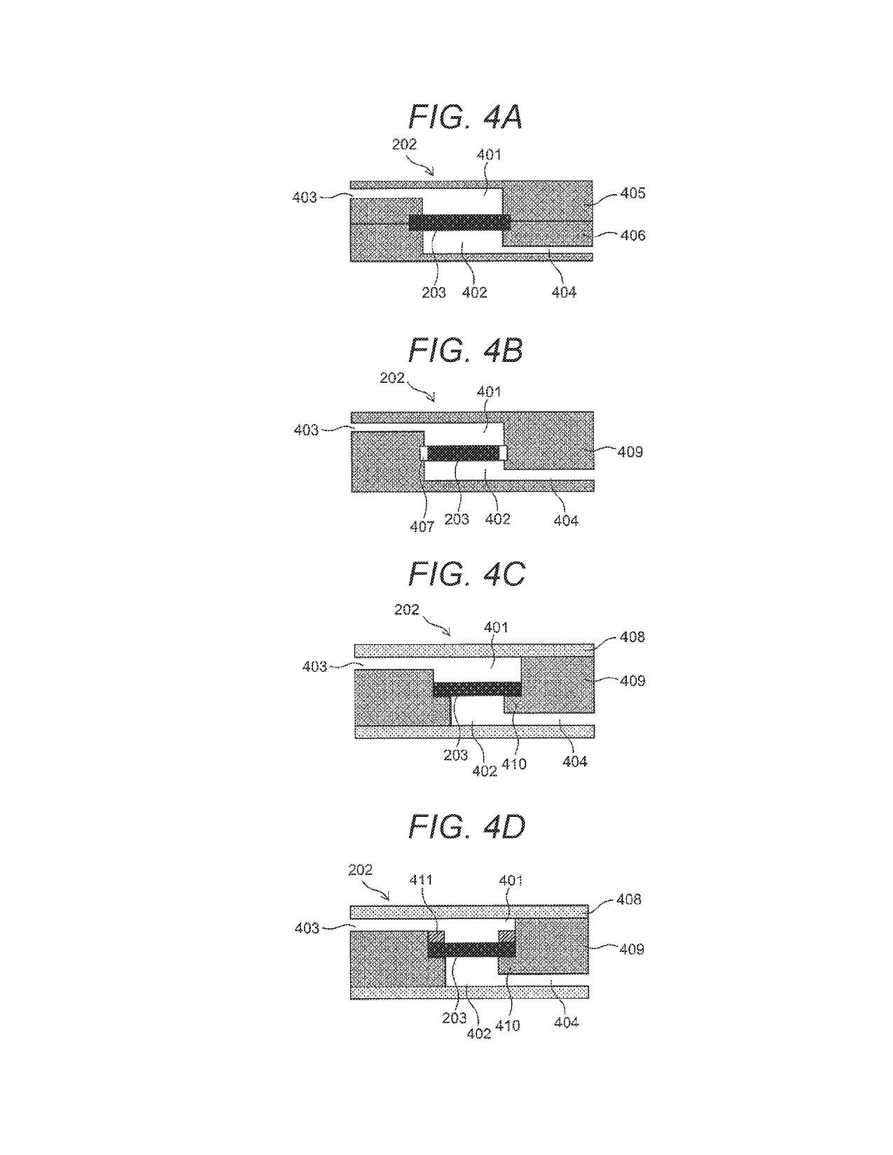

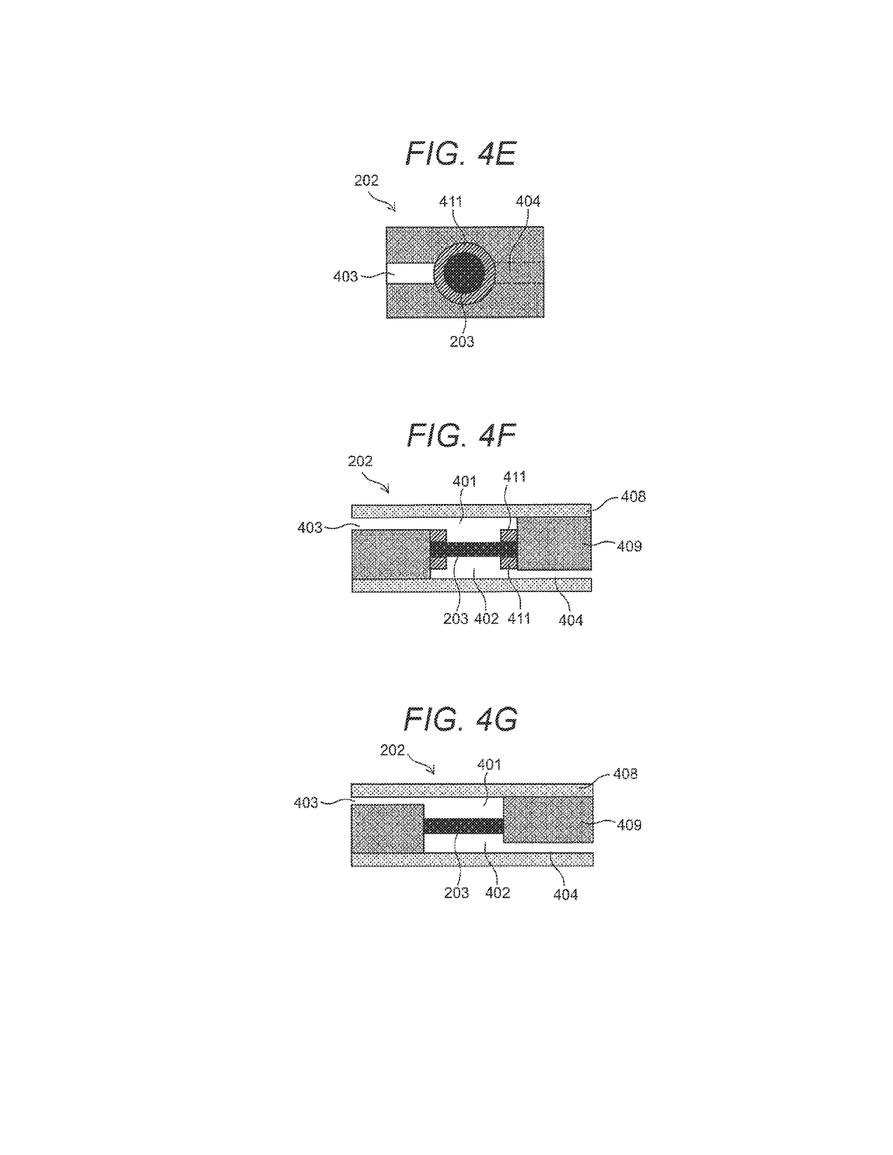

FIG. 4A is a cross-sectional view showing an example of a method for installing the membrane 203. In the example of FIG. 4A, the membrane 203 is installed in the nucleic acid capturing/amplification chamber 202, and is installed so as to separate the nucleic acid capturing/amplification chamber 202 into the first space 401 and the second space 402. The nucleic acid capturing/amplification chamber 202 comprises a first path 403 connected to a first space 401 and a second path 404 connected to a second space 402. When the dissolution product is flowed from the first path 403 to the second path 404, all the dissolution product can pass through the membrane 203, so that the nucleic acid can be captured with high efficiency.

As shown in FIG. 4A, the flow path substrate is composed of an upper substrate 405 and a lower substrate 406. The membrane 203 can be installed so as to be sandwiched between the upper substrate 405 and the lower substrate 406. The nucleic acid can be captured with high efficiency without the dissolution product passing through the gap between the membrane 203 and the flow path substrate. In addition, the area through which the solution can pass is identical between the upper part and the lower part, and the volume of the membrane 203 in the sandwiched part is small. For this reason, it is possible to bring the nucleic acid captured on the membrane 203 into amplification with high efficiency and to wash off the inhibitor with high efficiency at the time of washing. In addition, since the area in which the nucleic acid is captured and comes into contact with the amplification reagent is limited, the amplification inhibition is small.

FIG. 4B is a cross-sectional view showing an example of a method for installing a membrane 203. The membrane 203 is fixed to the flow path substrate 409 with an adhesive 407. The adhesive 407 may be a material that melts with heat, such as paraffin. The adhesive 407 may be a material that does not inhibit the amplification reaction. The structure of FIG. 4B can be manufactured at low cost because it is not necessary to bond the substrates to each other as in FIG. 4A.

FIG. 4C is a cross-sectional view showing an example of a method for installing a membrane 203. The flow path substrate 409 includes a pedestal 410 for fixing the membrane 203. The pedestal 410 plays a role of preventing the membrane 203 from coming off and generating a gap during solution conveyance. In addition, the film 408 is provided on the upper surface and the lower surface of the flow path substrate 409, and the first space 401, the second space 402, the first path 403, and the second path 404 are defined by the flow path substrate 409 and the upper and lower films 408. When the dissolution product is conveyed from the first path 403 toward the second path 404, the dissolution product does not leak into the gap between the membrane 203 and the flow path substrate 409, and thus the nucleic acid can be captured into the membrane 203 with high efficiency. The membrane 203 is preferably designed to be the same as or slightly larger than the diameter of the pedestal 410. The structure of FIG. 4C can be manufactured at low cost because it is not necessary to bond the substrates to each other as in FIG. 4A.

FIG. 4D is a cross-sectional view showing an example of a method for installing a membrane 203. The membrane 203 is disposed so as to be sandwiched between the pedestal 410 and ring 411. FIG. 4E is a top view of FIG. 4D. The dissolution product is not affected by the transport direction, and the nucleic acid can be captured by the membrane 203 with a high efficiency without leaking into the gap between the membrane 203 and the flow path substrate 409. Since the area capturing the nucleic acid and in contact with the amplification reagent is limited, the amplification inhibition is small. As the material of the ring 411, for example, materials that are not elastically deformed, such as polyester resins, polystyrene, polyethylene resins, polyamides, acrylic resins, fluororesins, polycarbonate resins, polyurethane resins, methylpentene resins, phenol resins, melamine resins, epoxy resins and vinyl chloride resins, can be used. The ring 411 may not be made of a resin. Specific physical properties of the ring 411 include a Young’s modulus of 5 MPa or more and 1 GPa or less, an elastic modulus of 100 kPa or more, and an elastic strain limit value of less than 100%, at an ordinary temperature, etc. The ring 411 may be a shape memory alloy or a shape memory resin. The ring 411 may be made of a material that melts by heat or ethanol. The structure of FIG. 4D can be manufactured at low cost because it is not necessary to bond the substrates to each other as in FIG. 4A.

FIG. 4F is a cross-sectional view showing an example of a method for installing a membrane 203. The membrane 203 is installed so as to be sandwiched between the two rings 411. By adopting such a structure, the thickness of the flow path substrate 409 can be reduced.

FIG. 4G is a cross-sectional view showing an example of a method for installing the membrane 203. The membrane 203 is installed so as to be fitted into a through hole of the flow path substrate 409. By adopting such a structure, a chip can be manufactured at low cost. However, for example, when a soft membrane such as a silica membrane is used, there is a possibility that the membrane may come off during chip conveyance and solution conveyance. For example, when a hard cellulose membrane, etc. having a thickness of 1 mm or more is used, it is preferable to adopt the present structure.

By the installation method of the membrane 203 shown in FIGS. 4A to 4G, a nucleic acid can be captured on the membrane 203 with high efficiency. However, the method of installing the membrane 203 is not limited to the above-mentioned method. The membrane 203 may be installed by a combination of a plurality of methods including the installation method described above.

### Sensitivity

Here, the quality of an amplified product of STR-PCR will be described. As used herein, a locus refers to a position of a gene on a chromosome. A typical kit for STR-PCR includes primers that can uniquely increase each locus.

As used herein, an allele refers to a genetic variant that can be distinguished on the same locus. In DNA identification, when the nucleic acid is derived from one person, there may be both a case where two alleles exist on the same locus (heterozygote) and a case where one allele exists (homozygote).

When sufficient nucleic acid quantities are present, the two peaks from the heterozygous loci show approximately the same height. When the nucleic acid quantities are insufficient, the probability that the amount of the nucleic acid derived from each gene becomes non-uniform increases, and the difference between the two peak intensities significantly increases. In addition, when amplification is excessive, a short nucleic acid is amplified preferentially to a long nucleic acid. The heights of the peaks derived from the same locus have large deviations because shorter nucleic acids are preferentially amplified. When a ratio of the two peak intensities increases, they cannot be distinguished from a stutter peak. In addition, it becomes difficult to assign a mixed sample. Therefore, in order to determine whether a significant CE analysis has been successfully performed, it is a reference that a ratio of the intensity of a smaller peak to the intensity of a larger peak (Peak to height ratio: PHR) in the two peaks is 10% or more, 40% or more depending on the case, or 60% or more.

The sensitivity is determined by the dissolution efficiency, the capture efficiency, the elution efficiency, the carry-in efficiency, the amplification efficiency, and the capillary electrophoresis device.

The dissolution efficiency refers to a ratio of the nucleic acid contained in the dissolution product that has been eluted and can be captured by the purification membrane among the nucleic acids contained in the introduced sample. Dissolution efficiency varies with the form of the sample, the dissolution time, the dissolution temperature, the dissolution reagent, the binding reagent, and the surface adsorption to the flow path substrate.

The capture efficiency refers to the ratio of the nucleic acids that can be captured by the membrane among the nucleic acids contained in the dissolution product. When there is a gap such that the dissolution product can enter the gap between the membrane and the flow path substrate, the capture efficiency decreases. In addition, when a thin membrane or a rough membrane is used, the capture efficiency decreases. When the membrane is thin or rough, the flow path resistance is small. In addition, when the storage state of the membrane is poor, or when an appropriate membrane is not selected, the ratio of activated sites (silanol groups in the case of the Boom method) capable of capturing a nucleic acid on the surface is low, and the capture efficiency decreases. In the case of a thick membrane or a fine membrane, the flow path resistance is large. In addition, the membrane having a high capture efficiency tends to adsorb substances essential for the amplification reaction, particularly polymerase. In addition, a membrane having a high capture efficiency tends to have a large flow path resistance.

Release efficiency refers to the proportion of nucleic acids released from the membrane when contacted with a low salt concentration solution among the captured nucleic acids. When the capture efficiency of the membrane is high, the elution efficiency tends to be low. In addition, the higher the temperature of the solution, the higher the elution efficiency. When nucleic acid is released from the membrane during washing and loss occurs, the apparent elution efficiency is low. When amplification together with a membrane is performed, it is not possible to strictly separate the elution efficiency and the amplification efficiency.

A magnification at which nucleic acid increases per cycle is referred to as an “amplification rate”. In ideal amplification, the amplification rate per cycle is 2. In addition, when an amount of a nucleic acid required to obtain an ideal profile in an ideal amplification reaction is 1 and an amount of a nucleic acid required to actually obtain an ideal profile is x, an amplification efficiency is represented by 1/x. When the sensitivity is determined by the PHR, the amplification efficiency is determined by the first few cycles. If the amplification rate of the first several cycles is close to 2, the amplification efficiency is almost 1 even if the amplification rate of the subsequent stage is less than 2.

A carrying-in efficiency refers to a proportion of nucleic acids subjected to an amplification reaction among released nucleic acids. When an elution step with an eluate is performed, the carrying-in efficiency can be defined, whereas when a reaction of amplification together with a membrane is performed, it can be basically regarded as 100%.

Even if dissolution, capture, release and amplification are performed with high efficiencies, the sensitivity decreases when the detection sensitivity of a capillary electrophoresis device is insufficient. In this case, the sensitivity can be increased by injecting more amplified product into the capillary electrophoresis device, increasing the number of amplification cycles, or increasing the amplification rate of each cycle. The amplification efficiency may also vary depending on the loci.

In general, when the amount of nucleic acid introduced into the STR-PCR is less than 20 copies, a peak balance may deteriorate or some peaks may not be detected due to a stochastic influence. Therefore, in order to perform the STR-CE analysis with high sensitivity, pretreatment that can maximize the amount of nucleic acid carried into the amplification unit is required.

In general purification, a nucleic acid captured on a membrane is released in an eluate and recovered as an elution product. When the liquid amount of the elution product is small, the elution efficiency is low. On the other hand, when the amount of the eluate is large, a large amount of amplification reaction liquid is required. When a large amount of amplification reaction liquid is used, the cost increases, the time required for temperature control increases, and the concentration of the amplified product decreases. In addition, since a part of the eluate remains in the membrane, the amount of the eluate varies. Nucleic acid purification by a spin column using a centrifuge, which is frequently used on a bench top, is not realistic because an apparatus becomes complicated and large when it is incorporated into a flow path device. However, in elution without centrifugation, the amount of liquid that can be recovered is smaller and more likely to fluctuate than on a bench top. Furthermore, the elution efficiency is also likely to vary. If the eluate in a varied amount is mixed with the amplification reaction liquid, the sensitivity is reduced. This is because the concentrations of reaction essential substances such as primers and dNTPs contained in the amplification reagent vary from their ideal states. Therefore, it is necessary to measure a certain amount of liquid and mix it with an amplification reaction liquid. However, when a certain amount is measured, an eluate to be discarded is generated. To summarize the above, by performing elution, loss of a nucleic acid occurs, and the sensitivity is lowered. When a general purification method is used, a product of a dissolution efficiency, a capture efficiency, an elution efficiency, and a carrying-in efficiency is 5% to 50%.

Inhibition occurs when hematin, humic acid, etc. are put into amplification. Furthermore, in the case of amplification together with a membrane, amplification inhibition constantly occurs depending on how the reagent is put into the membrane or the type of the membrane. When amplification inhibition occurs, the peak intensity and PHR decrease. Furthermore, not all loci are always equally amplified and inhibited, and only some loci may undergo significant amplification inhibition. When there is a significant difference in peak intensity between loci due to amplification inhibition, a peak having a larger peak intensity exceeds the upper detection limit or a peak having a smaller peak intensity falls below the lower detection limit in CE analysis. Under the same amplification condition, the analyzable initial DNA amount range is narrowed. As described above, when amplification inhibition occurs, the sensitivity decreases, or the peak intensities among loci significantly differ, and thus an analyzable DNA amount range is narrowed.

### Amplification Together with Membrane

When the membrane capturing the nucleic acid is brought into the nucleic acid capturing/amplification chamber 202, or when amplification together with a membrane is performed by capturing a nucleic acid by the membrane installed in the nucleic acid capturing/amplification chamber 202, the carrying-in efficiency is 100%. In addition, by bringing the membrane into contact with an amplification solution for a long period or heating the entire amplification solution, the elution efficiency can be brought close to 100%. In addition, depending on the type of the membrane, amplification can be performed in a state where nucleic acid is captured on the membrane, and thus the elution efficiency can approach 100%. In the case of amplification together with a membrane, a mechanism for metering the eluate and a mechanism for mixing the eluate with the amplification reagent are unnecessary, so that the configuration of the flow path device becomes simple and the cost of the flow path device is reduced.

Since the amplification reagent has a low salt concentration, the amplification reagent has an effect of releasing the captured nucleic acid similarly to the eluate generally used in the Boom method. In addition, when heated, the nucleic acid is released from the membrane with higher efficiency. Furthermore, the nucleic acid is released from the membrane with higher efficiency by not only contacting the reagent but also carrying so that the reagent passes through the membrane. The elution efficiency can be increased by allowing time after the contact. The nucleic acid may be amplified while being captured by the membrane. Amplification also occurs even while being captured.

### Details of Flow Path Device

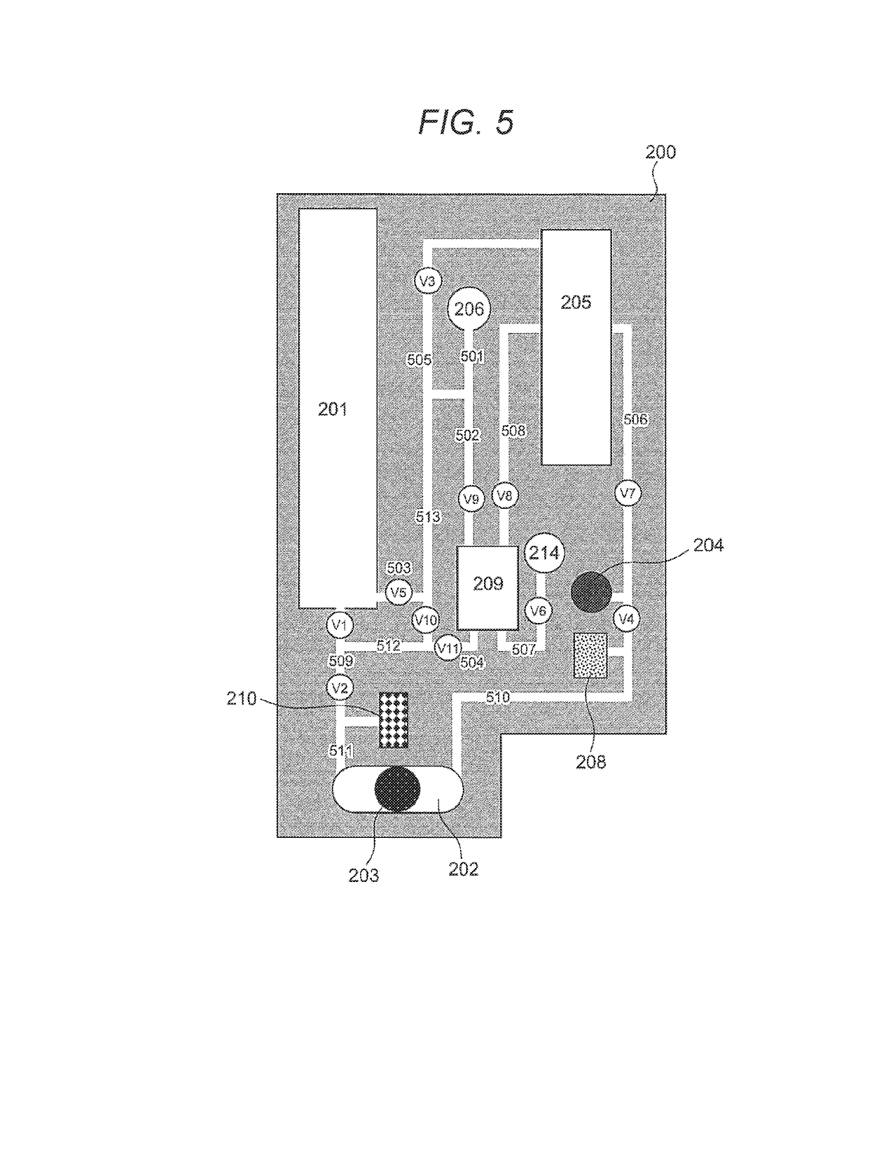

FIG. 5 is a top view showing a detailed configuration example of the flow path device 200 including the nucleic acid capturing/amplification chamber 202. The flow path device 200 includes external connection ports 206 and 214. The flow path device 200 can exchange a solution with the analysis system 1 via the external connection ports 206 and 214, and can receive control of air pressure. Here, it is assumed that the external connection port 206 is connected to a pump, and the external connection port 214 is connected to the detection unit 300.

The external connection port 206 is connected to a flow path 501. The flow path 501 is connected to a flow path 502 including a valve V9, 505 including a valve V3, and a flow path 513. The flow path 513 is connected to the flow path 503 including the valve V5, the flow path 512, and 504 including a valve V11. The flow path 503 is connected to a dissolution chamber 201. The flow path 502 is connected to a mixing chamber 209. The dissolution chamber 201 is connected to flow paths 509 and 512 provided with a valve V1. The flow path 509 is connected to a flow path 511 including a valve V2. The nucleic acid capturing/amplification chamber 202 is connected to a flow path 511 and a flow path 510 including a valve V4. The flow path 510 is connected to a flow path 506 including a valve V7 connected to a waste liquid tank. The flow paths 502 and 504 are connected to the mixing chamber 209. The mixing chamber 209 is connected to the external connection port 214 by a flow path 507 including a valve V6, and is connected to the waste liquid chamber 205 by a flow path 508 including a valve V8. A reagent tank 208 is connected to the flow path 510 between the valve V4 and the nucleic acid capturing/amplification chamber 202, and a reagent tank 210 storing the electrophoresis reagent is connected to the flow path 511 between the valve V2 and the nucleic acid capturing/amplification chamber 202. An air tank 204 is connected to the flow path 510 between the valves V4 and V7.

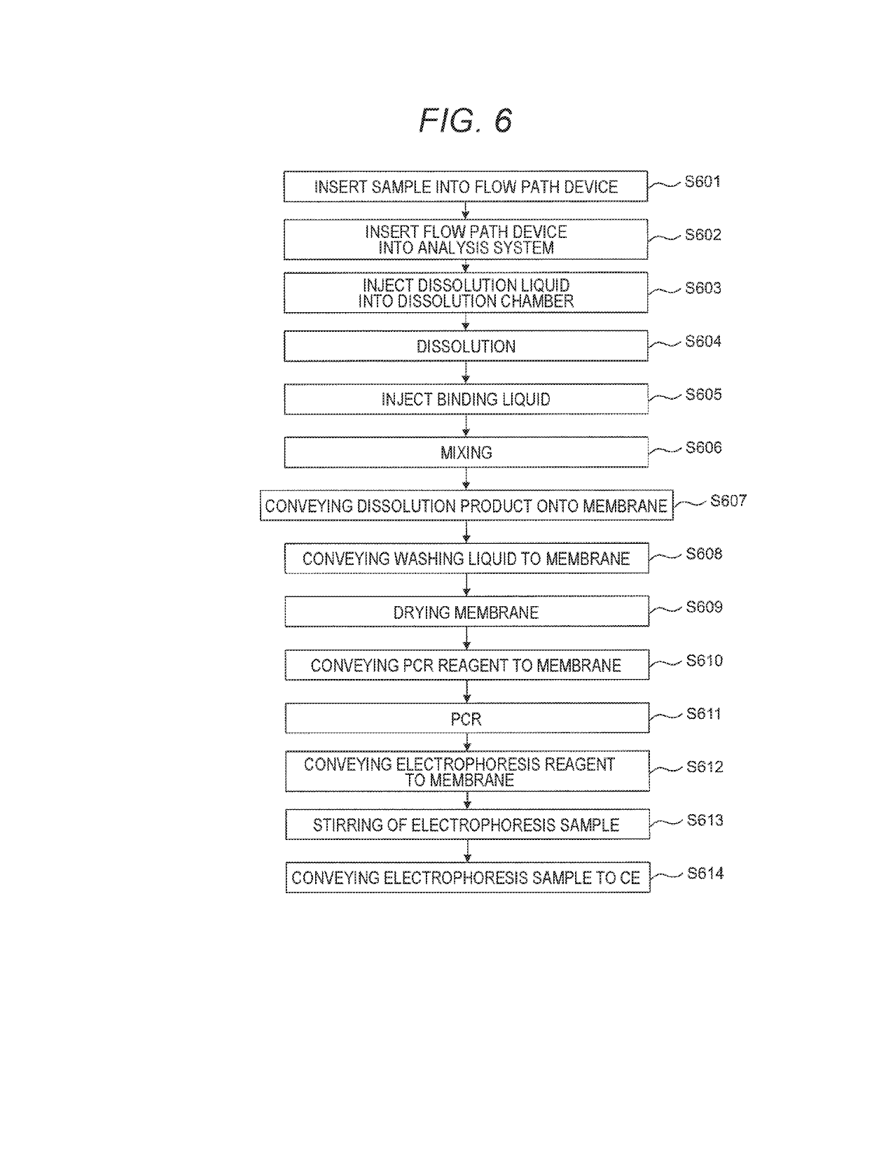

FIG. 6 is a flowchart illustrating an operation procedure in the flow path device 200. First, in step S601, the operator places a sample such as a swab into the dissolution chamber 201. Next, in step S602, the operator inserts the flow path device 200 into the installation location of the analysis system 1 and inputs an instruction to start the analysis.

In step S603, the processor 101 of the computer 100 closes the valves V1, V3, V9 and V10, and introduces the dissolution liquid from the external connection port 206 into the dissolution chamber 201 via the flow paths 501, 513 and 503. In step S604, the processor 101 may use the same pass to introduce air and agitate the dissolution liquid by bubbling to promote dissolution.

In step S605, the processor 101 introduces a binding liquid using the same path as in step 603. In step S606, the processor 101 uses the same path to introduce air and homogenize the dissolution product by bubbling.

In step S607, the processor 101 closes the valves V5, V10, V9, V8 and V11, and opens the valves V1, V2, V4, V7 and V3. Then, the processor 101 drives the pump 106 to make the external connection port 206 have a negative pressure, and conveys the dissolution product from the dissolution chamber 201 to the nucleic acid capturing/amplification chamber 202 via the flow paths 509 and 511. Thus, the nucleic acid in the dissolution product is captured by the membrane 203. The waste liquid of the dissolution product is conveyed to the waste liquid chamber 205 via the flow paths 510 and 506.

In step S608, the processor 101 closes the valves V3, V5, V1 and V11, and conveys the washing liquid from the external connection port 206 to the nucleic acid capturing/amplification chamber 202 via the flow paths 501, 513, 512, 509 and 511. The inhibitor remaining in the membrane 203 is conveyed to the waste liquid chamber 205 via the flow paths 510 and 506. In step S609, dry air is sent to dry the membrane 203. The dry air may be delivered at a positive pressure or negative pressure. The positive pressure has a lower risk of contamination, whereas the negative pressure can dry the washing liquid earlier.

In step S610, the processor 101 closes the valve V2, and releases the amplification reagent from the reagent tank 210 and conveys it to the nucleic acid capturing/amplification chamber 202. In step S611, the processor 101 closes the valve V2 and the valve V4 and performs an amplification reaction.

In step S612, when the amplification reaction is completed, the processor 101 opens the valves V2, V4, V11 and V8, closes the valves V1, V10 and V4, releases the electrophoresis reagent from the electrophoresis reagent tank 208, and conveys the amplified product to the mixing chamber 209 via the flow paths 511, 509, 512 and 504. Furthermore, the processor 101 closes the valve V7 and opens the valve V4 to push the amplified product from the air tank 204, and conveys the electrophoresis reagent and the amplified product left in the flow path to the mixing chamber 209.

If necessary, in step S613, the processor 101 may uniformly stir the electrophoresis reagent and the amplified product by closing the valves V3, V5, V1, V2 and V9, opening the valves V10 and V11, and bubbling the mixing chamber 209.

In step S614, the processor 101 opens the valve V6, closes the valves V8, V11, V3, V5 and V10, and conveys the electrophoresis sample in the mixing chamber 209 to a detection unit 300 (a capillary electrophoresis device) via an external connection port 214.

### Problem of Amplification Together with Membrane

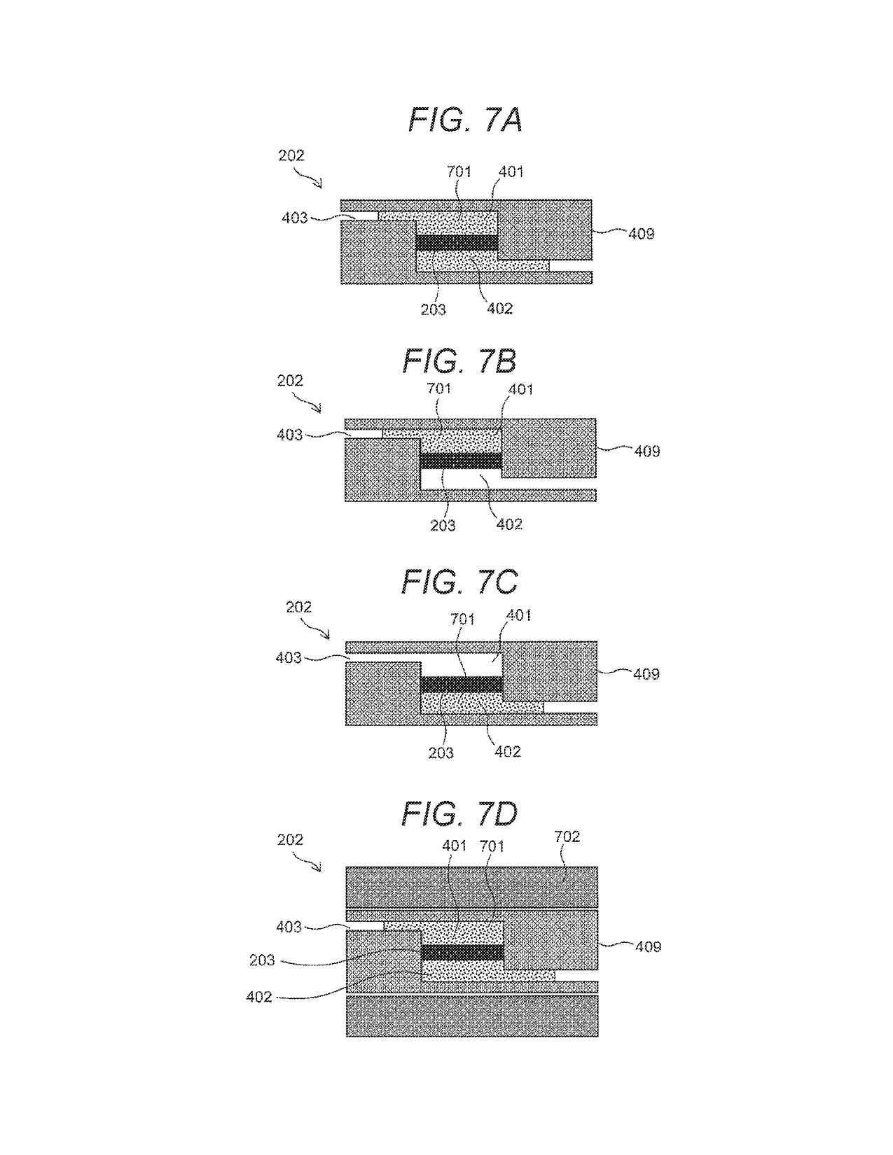

The membrane 203, which is capable of capturing nucleic acid with high efficiency, also tends to easily capture the components of the amplification reagent. FIGS. 7A to 7D are cross-sectional views each showing an example of a method for amplification together with a membrane in a nucleic acid capturing/amplification chamber 202. FIG. 7A shows an example in which an amplification reagent 701 is present in both the first space 401 and the second space 402 of the nucleic acid capturing/amplification chamber 202. The amplification reagent 701 is introduced from the first path 403. Therefore, the amplification reagent 701 existing in the second space 402 passes through the membrane 203.

FIG. 7B shows an example in which the amplification reagent 701 is present only in the first space 401 of the nucleic acid capturing/amplification chamber 202. The amplification reagent 701 is introduced from the first path 403. The amplification reagent 701 may stay in the first space 401 so as to be blocked by the membrane 203, or may stay in the first space 401 by being controlled using a liquid level detection sensor, a pressure sensor, etc. Only the surface of the membrane 203 facing the first space 401 may be wet with the amplification reagent 701, half of the membrane 203 may be wet, or the membrane 203 may be completely wet.

As shown in FIG. 7B, when the amplification reagent 701 is introduced from the first path 403 and the amplification reagent 701 is held, it is difficult to convey the liquid with good reproducibility if the membrane is hydrophilic. If the liquid amount is slightly small, the membrane 203 cannot be filled with the amplification reagent 701, and if the liquid amount is slightly large, the amplification reagent 701 moves across the membrane 203 to the second space 402. In addition, when the differential pressure of the pressure of the first space 401 with respect to the pressure of the second space 402 is slightly larger than the differential pressure necessary for establishing the state of FIG. 7B, the liquid goes beyond the membrane 203. Conversely, when the differential pressure is slightly lower, the liquid does not contact the membrane 203. When the liquid is not in contact with the membrane 203, the nucleic acid cannot be liberated from the membrane 203. On the other hand, when the liquid exceeds the membrane 203, a large amount of nucleic acid is liberated in the excess portion, but the amplification reagent 701 necessary for amplification is captured by the membrane 203 and cannot pass therethrough, so that amplification is inhibited.

FIG. 7C shows an example in which most of the amplification reagent 701 is conveyed so as to fill the second space 402. When the amplification reagent 701 passes through the membrane 203, the liquid feeding pressure needs to exceed the valve point of the membrane 203. In particular, when the membrane 203 is made of a hydrophilic material, a higher pressure is required for the amplification reagent 701 to escape than to pass through the membrane 203. Therefore, when constant pressure liquid feeding is performed at a low pressure of about 10 kPa, the amplification reagent 701 stops so as to be caught by the membrane 203.

FIG. 7D illustrates a configuration example in a case where nucleic acid is amplified using a heater 702. When the amplification reaction is performed by heating and cooling both surfaces of the nucleic acid capturing/amplification chamber 202 with the heater 702, the amplification reagent 701 is present on both sides of the membrane 203, so that the amplification reagent is likely to be uniformly warmed. In addition, by heating and cooling from both surfaces, the temperature can be uniformly adjusted, and the temperature can be quickly adjusted. In addition, since the membrane 203 can be completely wetted with the reagent, the nucleic acid captured inside the membrane 203 can be stably and efficiently released, and can be amplified with high efficiency.

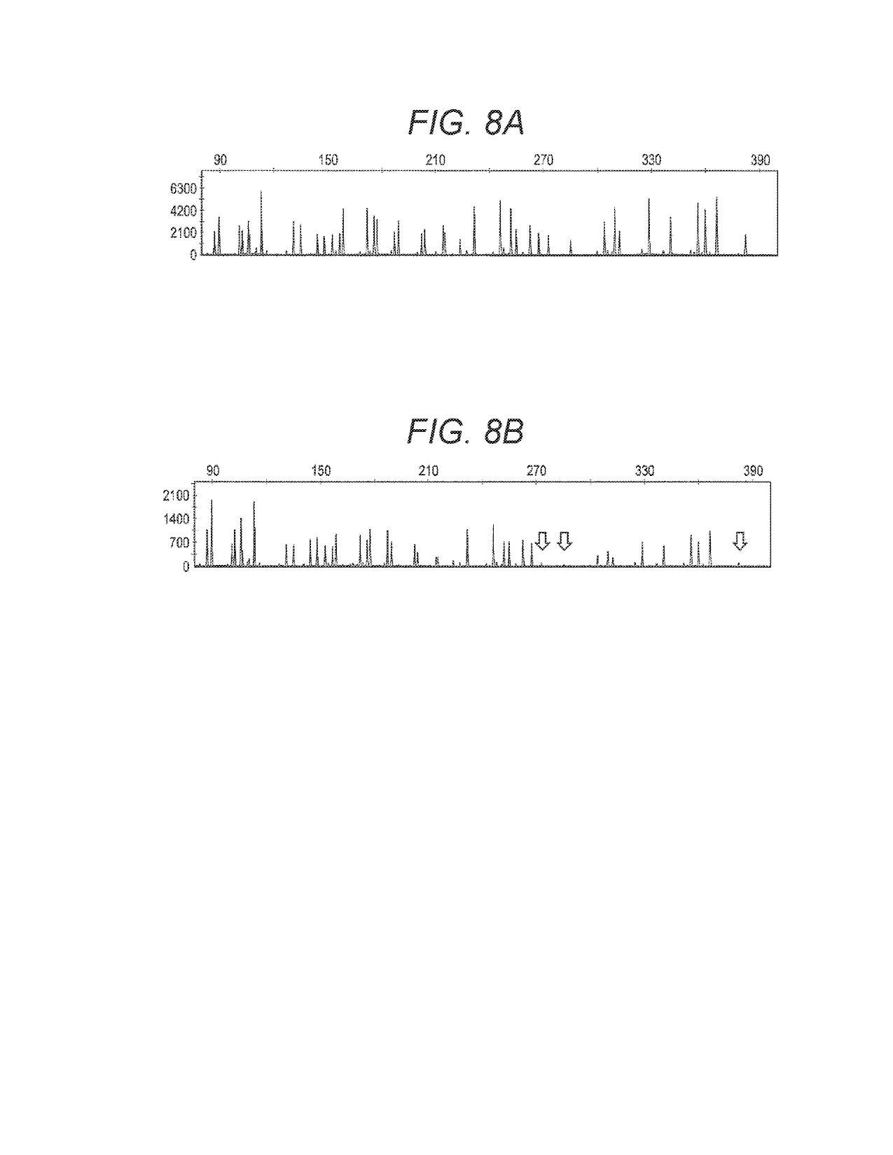

FIG. 8A is a diagram showing an example of a capillary electrophoresis profile of a normal STR-PCR amplified product. FIG. 8B is a diagram showing an example of a capillary electrophoresis profile of an amplified product obtained by passing an amplification reagent through the membrane 203 and amplifying a nucleic acid in a state where the amplification reagent after passing and the membrane 203 are mixed. As indicated by arrows in FIG. 8B, it can be confirmed that amplification is inhibited, and the intensity of some peaks is extremely low. This is because the component of the amplification reagent is captured by the membrane 203 by passing through the membrane 203, and the amount thereof is reduced, or the molecular structure of the component is denatured by being captured.

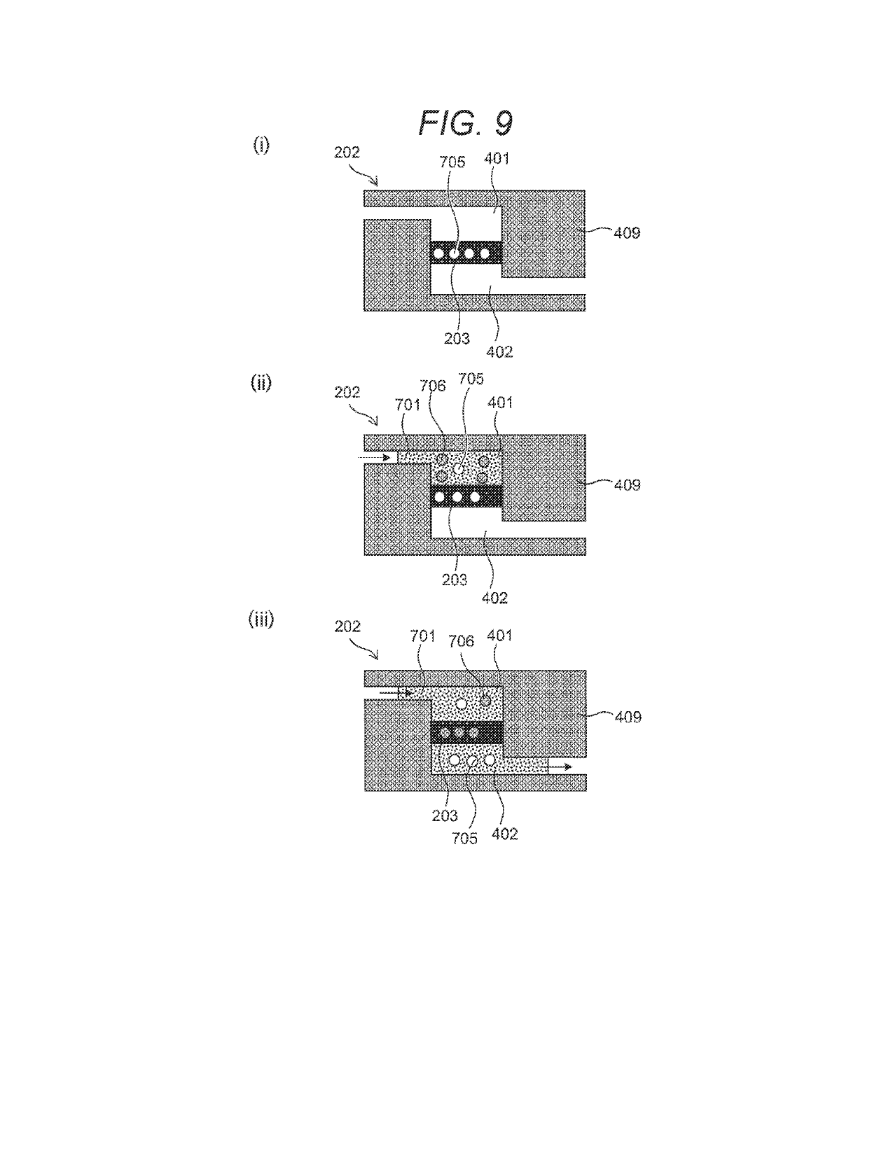

FIG. 9 is a cross-sectional view of the nucleic acid capturing/amplification chamber 202 for explaining the problem in the case of conveying an amplification reagent 701 as in FIGS. 7A and 7C. As shown in step (i) of FIG. 9, a nucleic acid 705 contained in the dissolution product is captured in the membrane 203. Thereafter, as shown in step (ii), when the amplification reagent 701 is introduced into the nucleic acid capturing/amplification chamber 202, the nucleic acid 705 is first released into the first space 401. Subsequently, as shown in step (iii), when the amplification reagent 701 enters the second space 402 across the membrane 203, more nucleic acid 705 is liberated by the amplification reagent 701 that has passed through the membrane 203. On the other hand, since an amplification component 706 contained in the amplification reagent 701 is captured by the membrane 203, the amplification component 706 is hardly contained in the amplification reagent 701 that has passed through the membrane 203. For this reason, although a large amount of the nucleic acid 705 is dissolved in the amplification reagent 701, the reaction does not proceed.

When heated for the amplification reaction, the components of the amplification reagent 701 captured by the membrane 203 are liberated. However, since it takes time for the components in the nucleic acid capturing/amplification chamber 202 to become uniform, the reaction time is long. The amplification reagent is locally depleted, the amplification efficiency is lowered, and the balance is lost. In addition, when dissociation and homogenization proceed in the middle of amplification, the PHR of the amplified product decreases and the sensitivity decreases due to a low amplification factor in the first several cycles and an unbalanced amplification.

One method for avoiding such a state is to increase the amount of reagent components. However, when the concentration of the reagent is increased to cope with the increase, there is an upper limit on the concentration that can be set. In the case of increasing the liquid amount, there are disadvantages such as temperature adjustment variation and a longer reaction time due to an increase in heat capacity and an increase in amplification chamber volume. In addition, a membrane that does not catch the components of the amplification reagent may be selected. However, the capture efficiency of the nucleic acid is low. In particular, shortened nucleic acids cannot be captured. In addition, the elution efficiency is low.

In the amplification reaction, when the viscosity of the liquid increases or convection and diffusion are inhibited, the amplification balance is lost or the amplification magnification is decreased, leading to a decrease in sensitivity. Since the density of the membrane 203 is high and the contents cannot move back and forth between the first space 401 and the second space 402, convection and diffusion inside the nucleic acid capturing/amplification chamber 202 are inhibited as compared with a case where there is no membrane.

A negative pressure or a positive pressure may be applied from the external connection port 206 using a syringe pump, etc. to stir the solution before and after the reaction and during the reaction. By doing so, the reaction can be uniformly performed, the elution efficiency of the nucleic acid can be increased, and the amplification efficiency can be improved. In addition, the concentration of the reaction component, the released nucleic acid, and the amplified nucleic acid becomes uniform.

However, when the density of the membrane 203 of the nucleic acid capturing/amplification chamber 202 is high, the flow path resistance is large, and a high differential pressure is required for stirring. In addition, if stirring is performed a plurality of times during amplification, the liquid moves to the outside of the nucleic acid capturing/amplification chamber 202, and the amplification efficiency may decrease.

There is a risk that the amplified product goes out of the flow path device 200 and contaminates the analysis system 1. Using a syringe pump only during the first few cycles of amplification can reduce such risks. In the denaturation step, since the internal pressure is particularly high, the liquid pops out unless the valve is closed. When stirring is performed for each amplification cycle, complicated valve switching is required. As a method other than the valve, pressurizing from both ends may be used, but it is difficult to control.

If air bubbles are caught on membrane 203 during stirring, the valve point pressure must be exceeded for the air bubbles to exceed membrane 203. However, in the case where stirring is performed without detecting air bubbles and performing feedback, the air bubbles stay while being caught on the membrane 203, and thus stirring cannot be performed as expected.

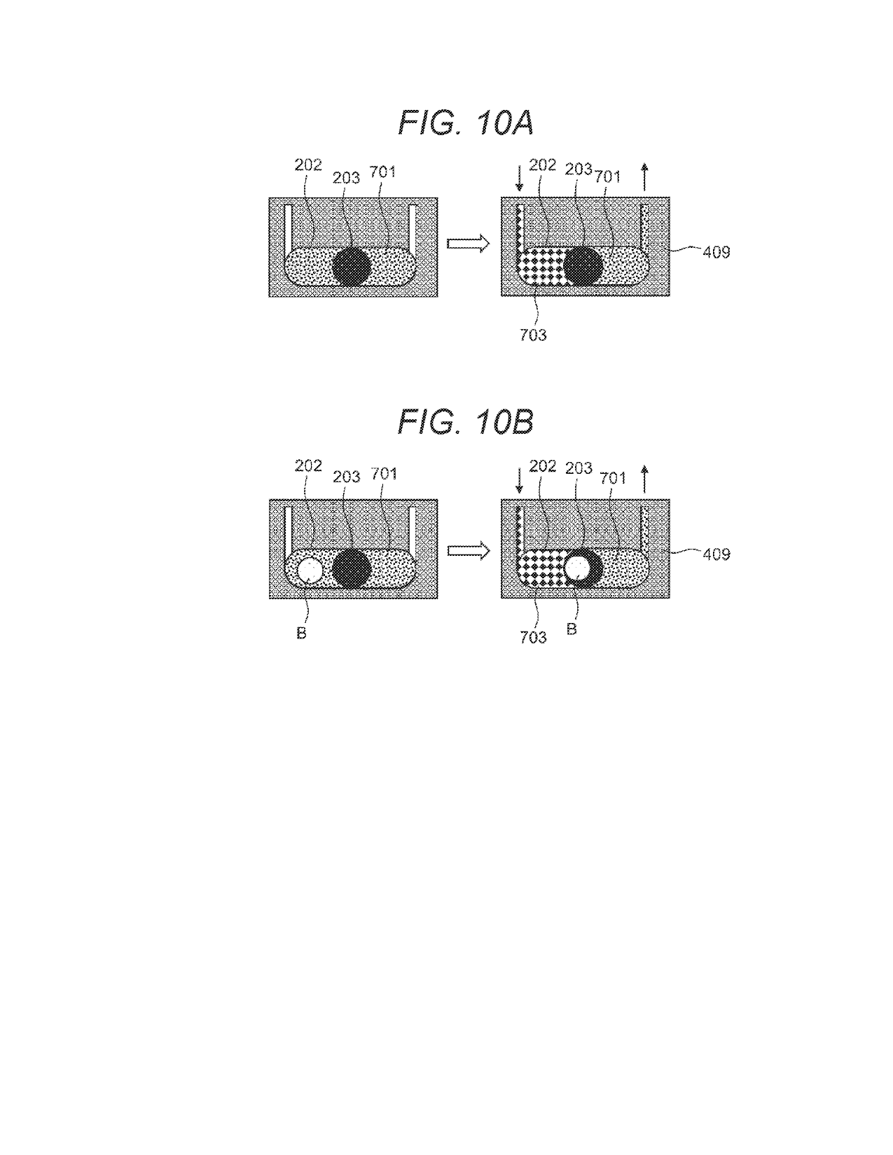

FIGS. 10A and 10B are top views for explaining the problem in conveying an amplified product from the nucleic acid capturing/amplification chamber 202. As shown in FIG. 10A, after the amplification reaction is ended, the amplified product is pushed out by the electrophoresis reagent 703 and conveyed to the mixing chamber 209. After the transfer, mixing is performed in the mixing chamber 209, and the electrophoresis sample is transferred to the detection unit 300.

However, as shown in FIG. 10B, air bubbles B are generated in the amplification chamber during the amplification reaction. The air bubbles B are generated when air remaining in the membrane 203 is released, or by air bubbles, water vapor, etc. remaining in the flow path device 200. When the air bubbles B exist in the nucleic acid capturing/amplification chamber 202, as shown in FIG. 10B, the air bubbles B are caught by the membrane 203 and liquid feeding is impossible. Although there is a possibility that the liquid feeding can be performed if a higher pressure is applied, there is no reproducibility in the generation of the air bubble B, and thus a mechanism for applying feedback using a liquid level detection sensor, etc. according to the generation situation of the air bubble is required, which is complicated.

In addition, when the electrophoresis reagent 703 is introduced, air bubbles are generated between the electrophoresis reagent 703 and the amplified product. Accordingly, since the membrane 203 captures the air bubbles, a high differential pressure is similarly required for conveyance. When the bubbles B passes through the membrane 203, the bubbles become fine bubbles, and the remaining air bubbles cause fluctuations in volume around the mixing chamber 209 at the subsequent stage. In addition, by being conveyed to the detection unit 300, it hinders sample injection into the capillary electrophoresis device, leading to a decrease in sensitivity and instability of operation. Furthermore, a high differential pressure is required also when the electrophoresis reagent 703 is removed from the membrane. Accordingly, since the membrane 203 captures the air bubbles, a high differential pressure is similarly required for conveyance. When a high differential pressure is required, the required performance of the valve increases. In addition, there is a risk that the liquid splashes unexpectedly and contaminates the device.

In summary, in amplification together with a membrane using a purification membrane capable of capturing a nucleic acid with a high efficiency, the nucleic acid contained in the sample can be maximally amplified, and the sensitivity can be increased. On the other hand, the conveyance is not stable. In addition, control by a complicated liquid level detection sensor, etc. is required, which results in high cost. Furthermore, since the amplification component is adsorbed to the membrane 203, amplification inhibition occurs, amplification cannot be performed in a well-balanced manner, and stirring and diffusion/convection are inhibited. As described above, the sensitivity is rather lowered by conducting amplification together with a membrane.

### Third Path

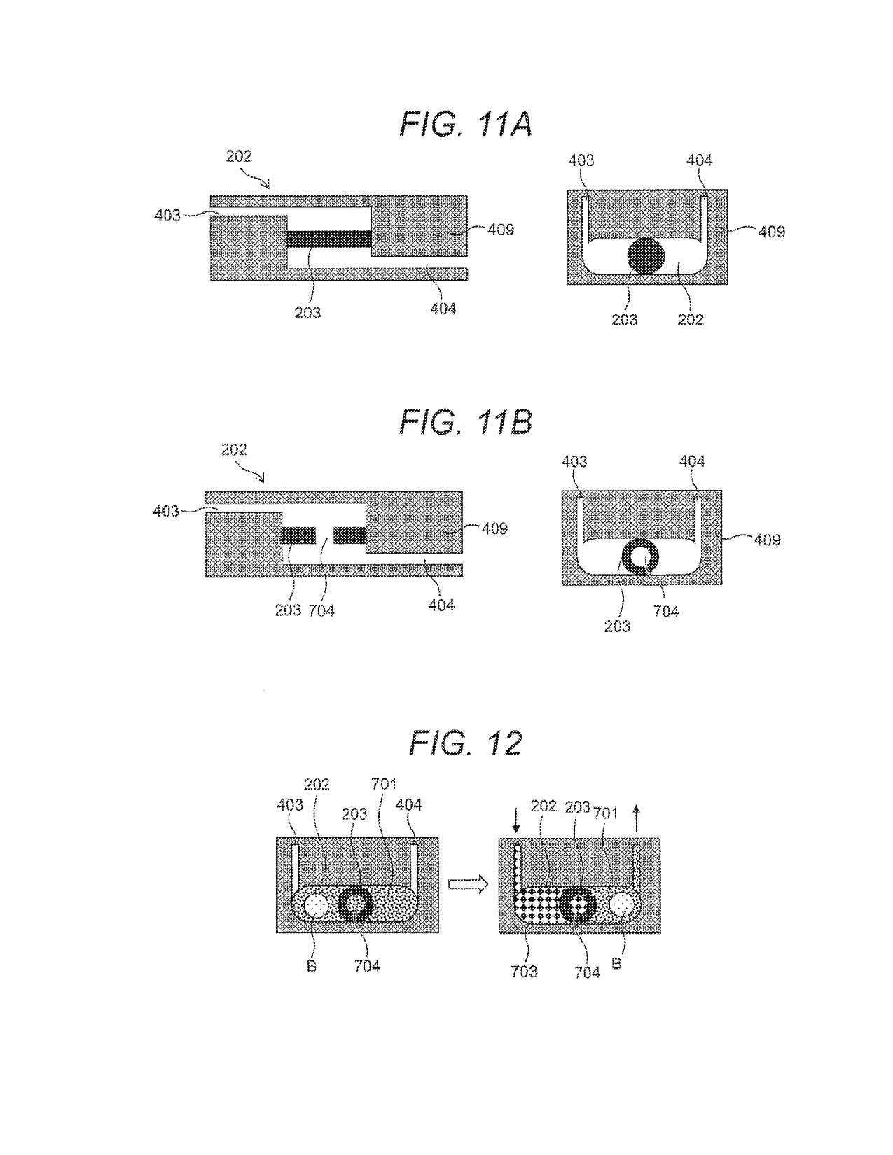

FIGS. 11A and 11B are views showing a method for forming a third path in the nucleic acid capturing/amplification chamber 202 of the present disclosure for solving the above-mentioned problem. As described above, the flow path device 200 includes the membrane 203 that captures nucleic acid contained in a sample, the nucleic acid capturing/amplification chamber 202 that defines spaces (the first space 401 and the second space 402) in which liquid is accumulated before and after the membrane 203, and the first path 403 and the second path 404 connected to the spaces. In the nucleic acid capturing/amplification chamber 202, a capture step of transporting a liquid containing nucleic acid from the first path 403 to the second path 404 and capturing the nucleic acid on the membrane 203, and an amplification step of amplifying the nucleic acid together with the membrane 203 using the membrane 203 in the flow path and the liquid in the space, are performed after the capture step. As shown in FIG. 11A, during the capturing step, a first space 401 close to the first path 403 and a second space 402 close to the second path among the spaces are separated by a membrane 203. As illustrated in FIG. 11B, a third path 704 through which liquid or gas (fluid) can move between the first space 401 and the second space 402 is newly formed after the capturing step is completed and before the amplification reaction is completed.

The flow path resistance of the third path 704 can be equal to or less than the flow path resistance of the membrane 203. The area of the third path 704 can be, for example, 0.01 mm² or more. The area of the third path 704 can in particular be one-tenth or more of the area of the membrane 203.

The third path 704 may be a single hole or may be divided into a plurality of portions. The third path 704 may exist at the start of use of the flow path device 200, and the liquid may be prevented from flowing by a plug, a valve, etc., or may be newly formed in the middle of use.

### Effect of Third Path

FIG. 12 is a view for explaining an effect of eliminating clogging by the third path 704. FIG. 12 illustrates a situation in which liquid feeding can be performed stably even air bubbles B are generated in the nucleic acid capturing/amplification chamber 202, by providing the third path 704. The air bubbles B generated in the first space 401 can move to the second space 402 through the third path 704. Therefore, when the electrophoresis reagent 703 is extruded from the first path 403 or the second path 404 and introduced into the nucleic acid capturing/amplification chamber 202, and the amplified product is recovered from the second path 404 or the first path 403, the amplified product can be smoothly recovered. Also, when the amplified product is recovered from the second path 404 or the first path 403 by pressurizing the first path 403 or the second path 404, the amplified product can be smoothly recovered. At this time, the amplified product may be recovered by negative pressure. However, when recovery is performed at a negative pressure, since an aspiration pump is contaminated with the amplified product, there is a risk of contamination between measurements.

The cause of clogging of the membrane 203 is not limited only to air bubbles. For example, also in a case where dust contained in the dissolution product is clogged in the membrane 203, the clogging can be similarly eliminated by the dust passing through the third path 704. Furthermore, the third path 704 may also allow a fluid to pass through as an alternative path if the meshes of the membrane 203 are clogged with air bubbles or dirt.

In order to obtain the effect of air bubble removal or unclogging by the third path 704, it is necessary that the hole diameter of the third path 704 is coarser than the density of the membrane 203 and the valve point is low. In particular, the hole diameter of the third path 704 can be 10 or more times the hole diameter or average hole diameter of the membrane 203. In addition, if the flow path resistance of the third path 704 is equal to or less than the flow path resistance of the membrane 203, it can be expected to be used as an alternative path.

By providing the third path 704, the flow path resistance of the nucleic acid capturing/amplification chamber 202 can be reduced. Therefore, the solution in the nucleic acid capturing/amplification chamber 202 can be stirred with a slight differential pressure. In addition, since the reagent existing in the first space 401 and the second space 402 can be moved back and forth via the third path 704 without crossing the membrane 203, diffusion and convection are likely to occur, and the components are uniform. In addition, the elution efficiency is improved. The area of the third path 704 needs to be one-tenth or more of the area of the membrane 203 in order to provide the third path 704 to obtain an effect of reducing the flow path resistance and an effect of facilitating diffusion and convection.

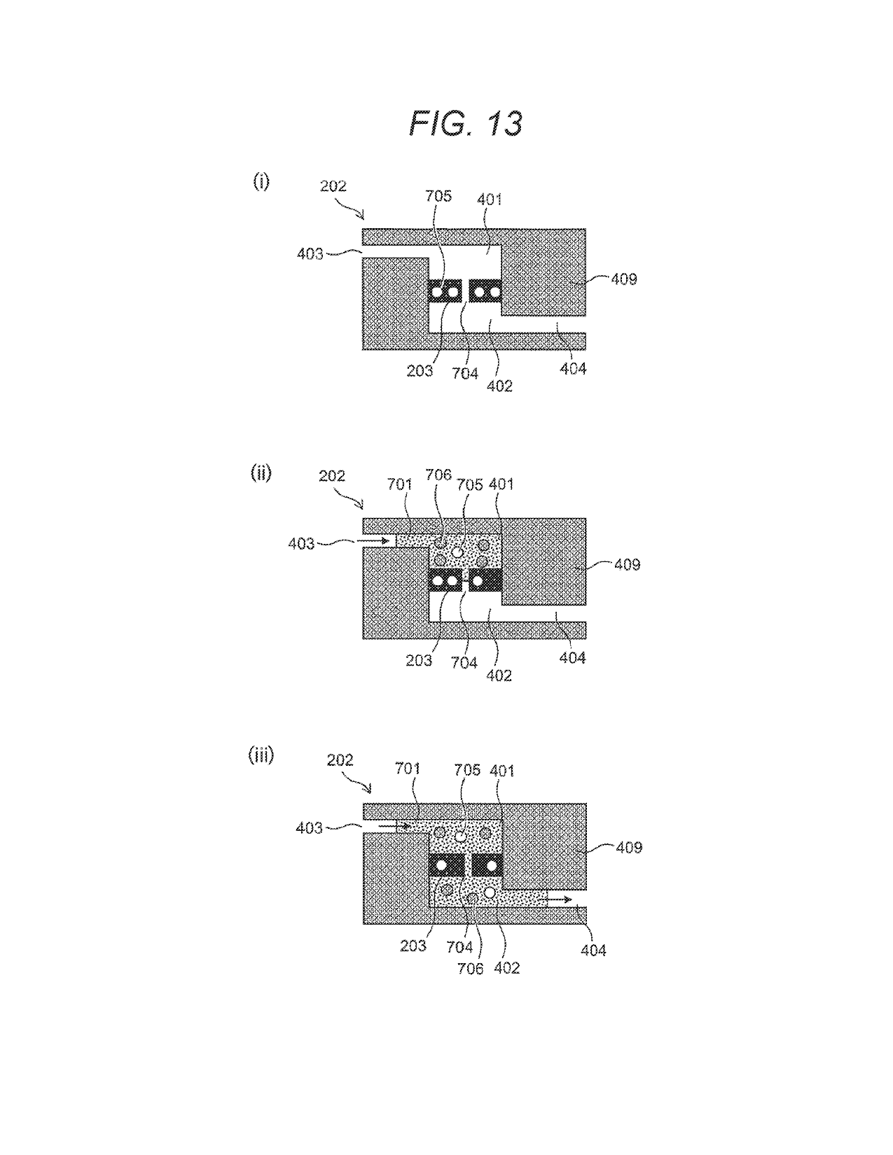

FIG. 13 is a view for explaining an effect of preventing component bias by the third path 704. FIG. 13 illustrates a situation in which the deviation of components in the nucleic acid capturing/amplification chamber 202 is improved by providing the third path 704. As shown in step (i) of FIG. 13, a third path 704 is provided after capturing the nucleic acid 705 on the membrane 203. Subsequently, as shown in step (ii) of FIG. 13, the amplification reagent 701 is introduced from the first path 403. When the membrane 203 contacts the amplification reagent 701, the nucleic acid 705 begins to be released. Subsequently, as shown in step (iii) of FIG. 13, most of the amplification reagent 701 starts to move to the second space 402 via the third path 704 having a smaller flow path resistance than the membrane 203. Since most of the amplification reagent 701 moves to the second space 402 via the third path 704 having a smaller flow path resistance than the membrane 203, the amplification component 706 is not caught by the membrane 203. The elution efficiency of the nucleic acid is lower than that in the case of step (iii) in FIG. 9, but the nucleic acid is gradually released when heated. The mixture may be stirred to promote releasing.

### Method for Forming Third Path

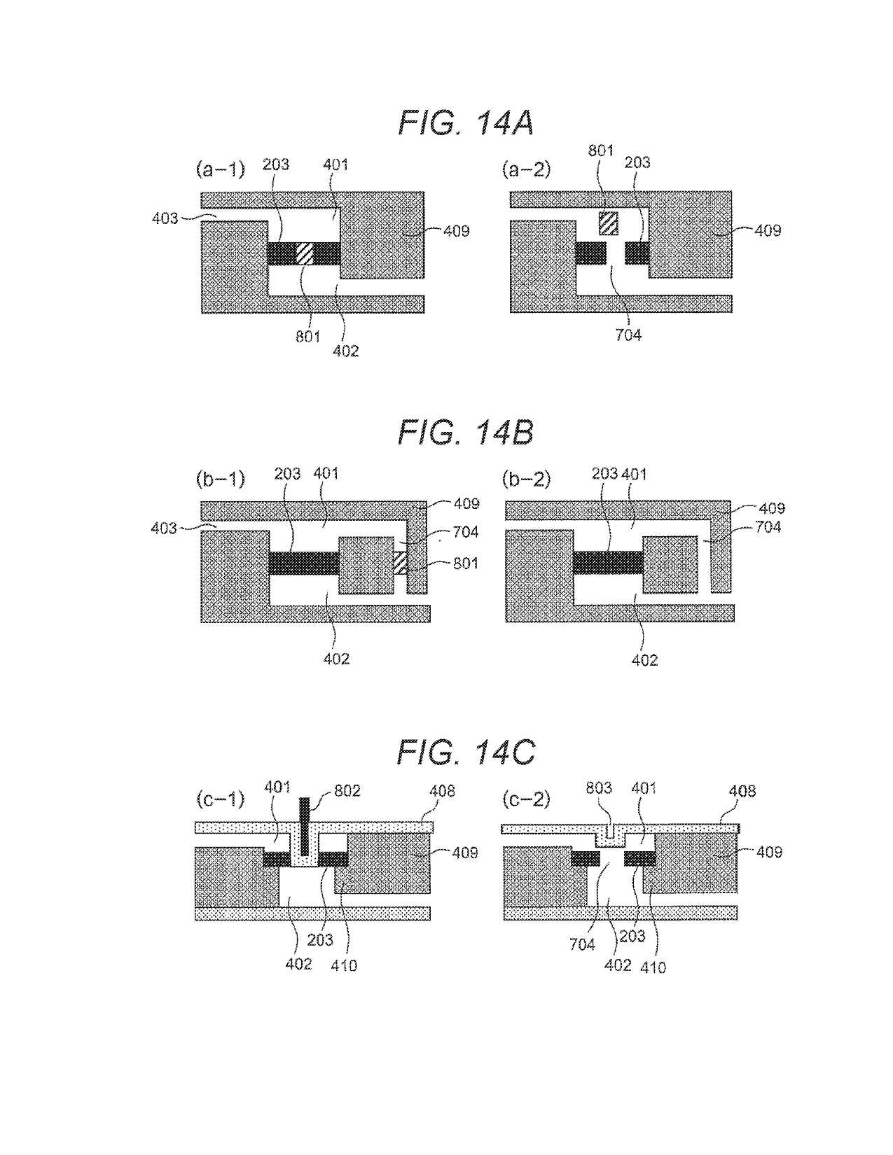

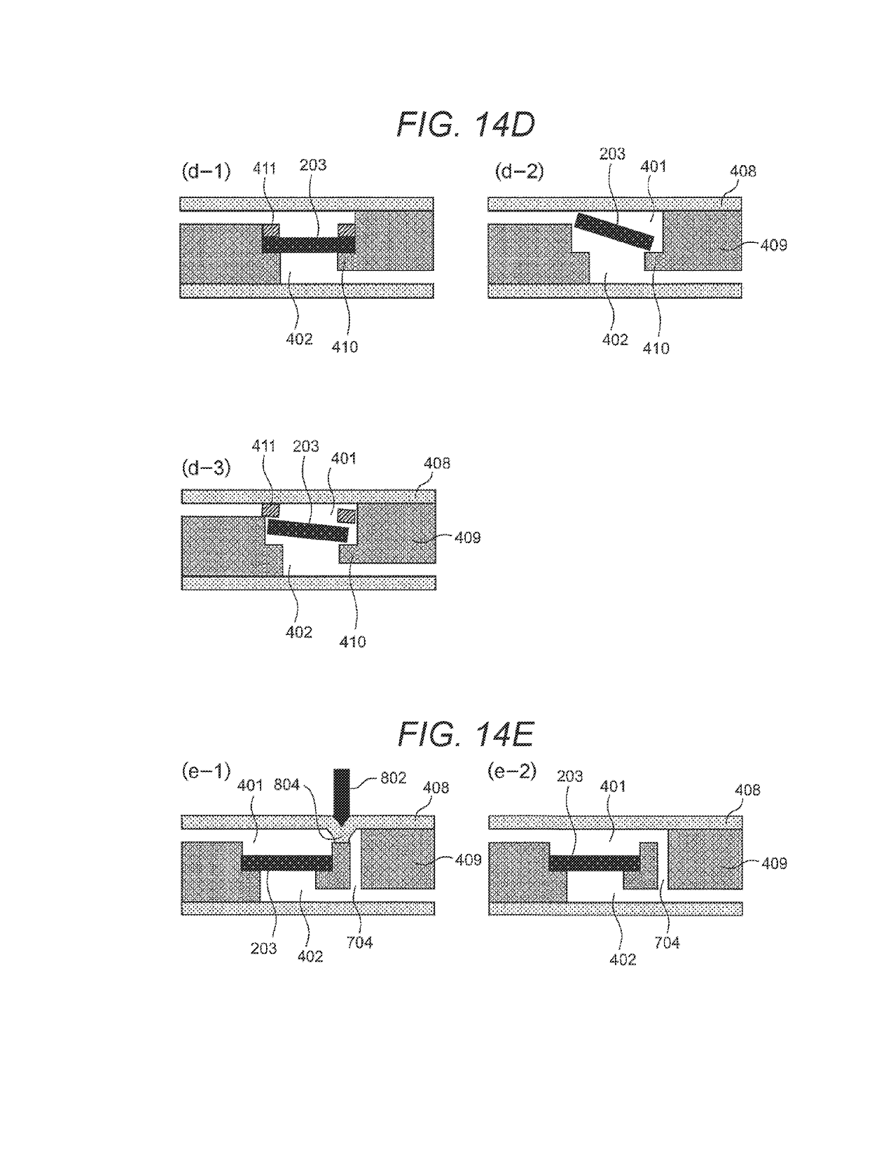

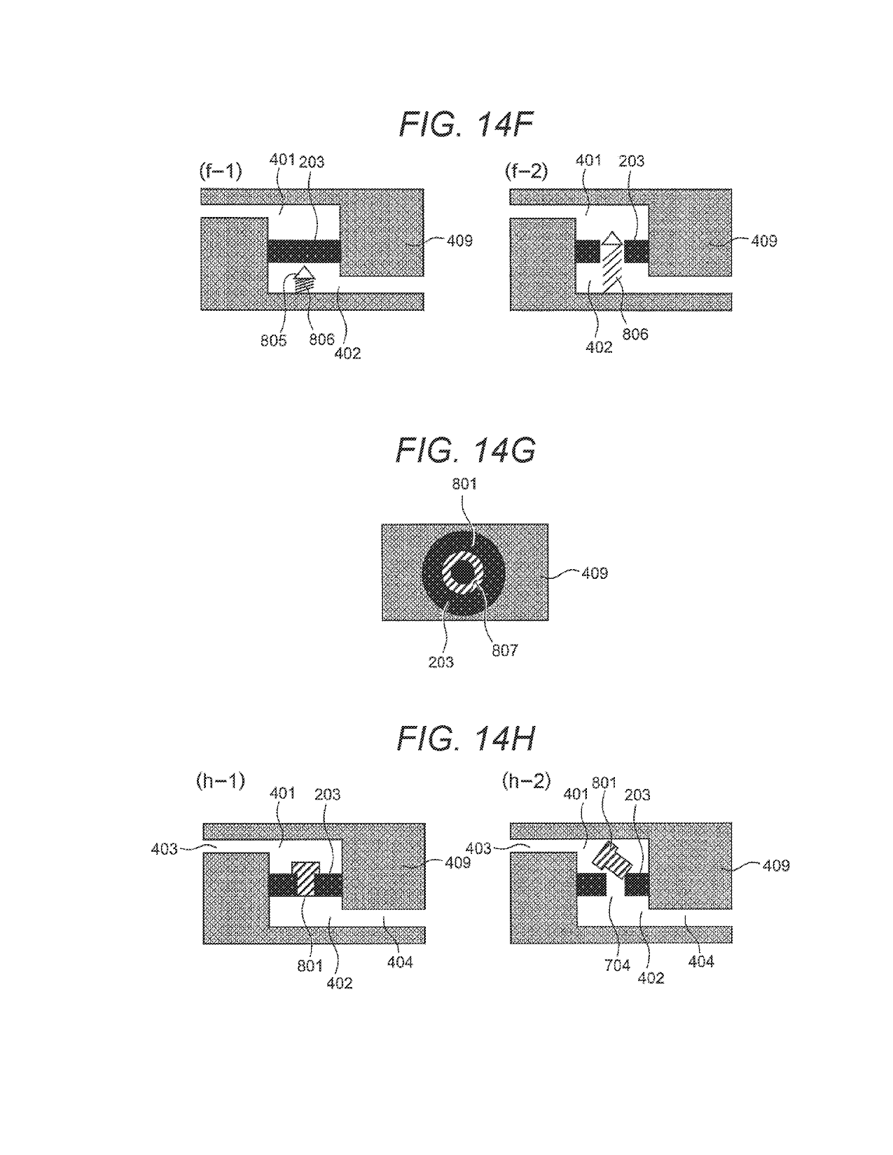

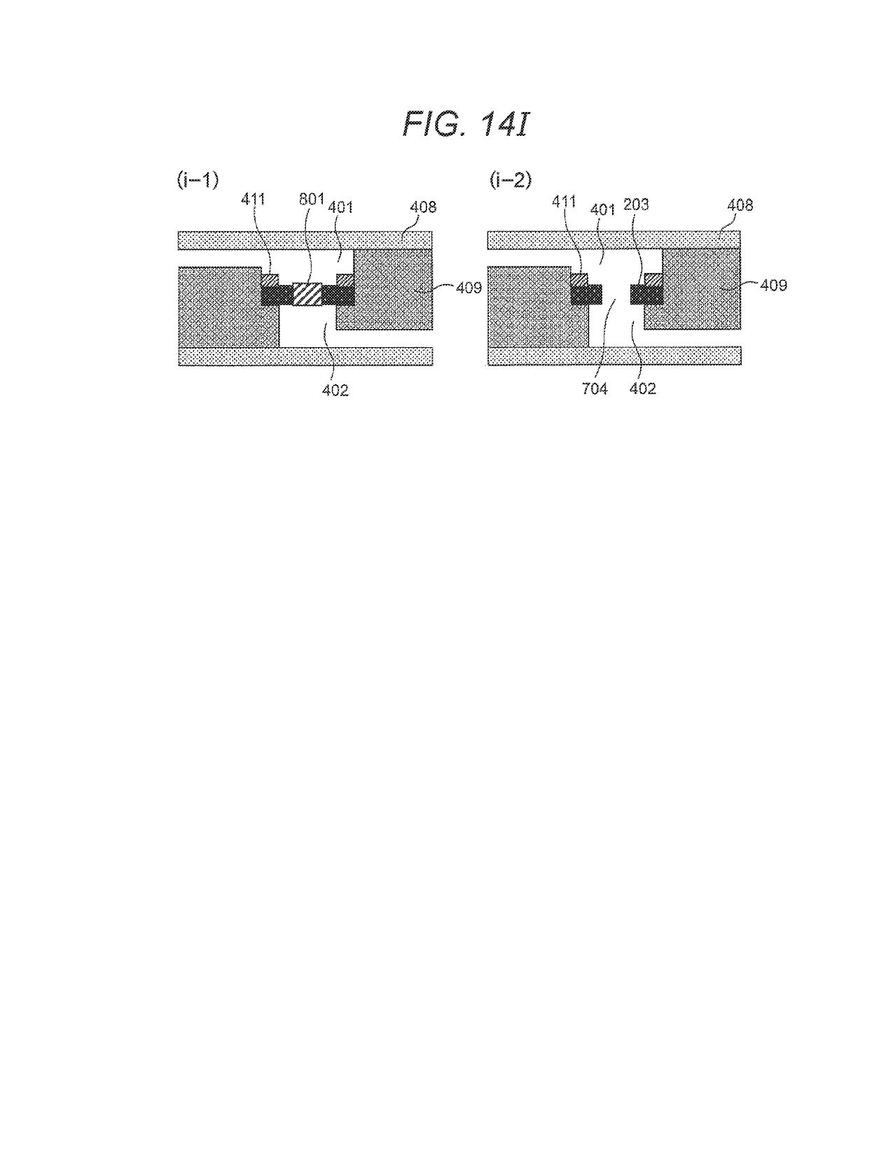

FIGS. 14A to 14I are views illustrating an example of a method for forming the third path.

(a-1) of FIG. 14A illustrates a configuration example in which the third path 704 exists inside the membrane 203, and the third path 704 is closed by the plug 801. The material of the plug 801 can be, for example, a water-soluble material, an ethanol-soluble material, or a heat-soluble material. Specifically, the material of the plug 801 may be, for example, paraffin, PEG, agarose, shape memory plastic, or shape memory alloy. Further, a part of the plug 801 may be made of a material having the above material and characteristics.

The material that melts with heat can be a material that melts at 50°C or higher, such as paraffin or PEG. The molecular weight and the molecular structure are appropriately selected in accordance with the timing of removing the plug 801. When heating occurs during dissolution, the temperature of the dissolution product passing through the membrane 203 is on the order of 40°C. When the plug 801 is made of a material that melts at 50°C or less, the plug 801 is removed from the dissolution product, and the capture efficiency is reduced. By using a material that is insoluble in an aqueous solution, such as paraffin, the influence on the reaction can be minimized. In addition, a material that is water-soluble and does not affect PCR as long as it is in a small amount, such as PEG, may be used. By using such a material, the material is not solidified even if the liquid temperature is lowered after the end of the PCR reaction, and thus the amplified product is not adversely affected when being transported out of the PCR chamber. In addition, being nonionic molecules, injection into the capillary is not affected adversely.

In the amplification reaction, the nucleic acid capturing/amplification chamber 202 is heated to 90°C or higher. It is also possible to select a material from which the plug 801 is detached at this time. In order to unplug the plug 801, UV irradiation may be performed.

(a-2) of FIG. 14A illustrates a state in which the plug 801 is detached and the third path 704 is opened. Until the third path 704 is opened, the flow path resistance of the flow path device 200 shown in FIG. 5 is the largest in the membrane 203. Therefore, when a pressure sensor is provided outside the flow path substrate and the path passes through the membrane 203, the differential pressure generally reflects the differential pressure generated in the first space 401 and the second space 402. The conveying speed of the syringe pump and the output of the diaphragm pump may be controlled. The plug 801 may be removed by providing a differential pressure between the first space 401 and the second space 402. Since a differential pressure of 30 kPa or more is required for conveying the dissolution product, the capture efficiency of the nucleic acid decreases when the plug 801 is disengaged at a differential pressure of 30 kPa at the solution temperature of the dissolution product. Therefore, the pressure at which the plug 801 is disengaged can be 50 kPa or more. After washing, during drying of the membrane 203, a high differential pressure is generated in the membrane 203 when air is blown by a diaphragm pump while the membrane 203 is wet. The plug 801 may be removed using the differential pressure at this time.

FIG. 14B illustrates a configuration example in which the third path 704 is installed in a place other than the membrane 203. Also in the configuration of FIG. 14B, the third path 704 is closed with a plug 801 as in FIG. 14A. When installing the third path 704 in this manner, the third path 704 can be used as an alternative path when the membrane 203 gets stuck.

FIG. 14C illustrates an example in which the third path 704 is formed inside the membrane 203 by an external force. At least one surface of the flow path substrate 409 is configured by a film 408. As the film 408, a material that is easy to stretch, specifically, a material having a breaking strain of 30% or more can be used. As the film 408, for example, polypropylene or rubber is used.

The external force is transmitted through the head 802 and deforms with the film 408 to form a third path 704 in the membrane 203. In the case of this method, the opening timing can be controlled more accurately than in the case of opening by heat. When the film 408 is made of a plastically deformable material such as polypropylene, a plastic deformation mark 803 remains. In the case of heating from both sides, a gap is generated due to plastic deformation, and thus the heat transfer coefficient decreases. In addition, the head 802 should be disposed or driven so as not to interfere with the heater.

FIG. 14D illustrates a configuration example in which the membrane 203 is fixed to the flow path substrate 409 with a ring 411 that melts or deforms and comes off by heat or a solution. In such a configuration, a gap corresponding to the third path 704 can be formed by removing or melting the ring 411 at the time of heating or solution conveyance.

As in FIG. 14D (d-1), when the ring 411 is compressing and pressing against the membrane 203, the ring 411 experiences a force in the upward direction in the drawing that causes the membrane 203 to try to return to its original position. At room temperature, frictional force generated between the outer wall of the ring 411 and the inner wall of the flow path substrate 409 allows the ring 411 to be held on the pedestal 410 of the membrane 203. On the other hand, when the elastic modulus becomes, for example, 1000 MPa or less at the time of heating, no frictional force is generated between the outer wall of the ring 411 and the inner wall of the flow path substrate 409, and the ring 411 is released by receiving the upward force from the membrane 203. Since the ring 411 once detached does not return even after cooling, the third path 704 can be stably formed.

The ring 411 may be made of a material having an elastic modulus of, for example, 1000 MPa or less at normal temperature. In a case where liquid feeding and air feeding such that the pressure in the first space 401 is higher and the pressure in the second space 402 is lower are performed, the ring 411 is not detached. On the other hand, when liquid feeding and air feeding are performed so that the pressure becomes higher in the second space 402, the ring 411 is detached at a certain differential pressure or more. For example, the ring 411 can be removed by generating a differential pressure of 30 kPa or more. Depending on the material of the ring 411, the material of the flow path substrate 409, the temperature at the time of use, the geometry of the ring 411 and the flow path substrate 409, etc., it is possible to arbitrarily set the degree of differential pressure at which the ring 411 can be removed.

FIG. 14E illustrates a configuration example in which the valve 804 is installed in a portion branching into the third path 704. The valve 804 is closed by deforming the film 408 with the head 802 and pressing the film against the flow path substrate 409. By removing the pressing of the head 802, liquid can be moved back and forth between the first space 401 and the second space 402 via the third path 704. As such, the third path 704 is installed at a location other than the membrane 203, and the third path 704 is closable at the valve 804.

FIG. 14F illustrates a configuration example in which the spring 806 including the protrusion 805 is provided in the second space 402. The spring 806 is made of a shape memory alloy, etc. By the heating, the spring 806 extends, and the protrusion 805 breaks the membrane 203 to form the third path 704. Unlike FIG. 14 C, since no external force is required, the third path 704 can be formed regardless of the material of the flow path substrate 409. In addition, since there is no need to consider plastic deformation of the film 408 or interference between the head 802 and the heater, the degree of freedom is high. The spring 806 may be provided in the first space 401.

FIG. 14G illustrates an example in which a part of the plug 801 is made of a material having a characteristic of being melted or deformed by heat. In the plug 801, a portion in contact with the membrane 203 may be made of a material having a characteristic of being melted or deformed by heat, and a portion of the core 807 may be made of a material that is not melted or deformed. By adopting such a configuration, it is possible to reduce the amount of material that melts or deforms while widening the cross-sectional area of the third path 704, so that the influence on the reaction can be minimized. The plug 801 may be divided into a plurality of parts when the third path 704 is formed. For example, the core portion of the plug 801 may be composed of a plurality of grains, and the core portion may be discrete when a material that melts or deforms melts or deforms. By adopting such a form, the formation of the third path 704 can be smoothly promoted.

FIG. 14H illustrates a configuration example in which the third path 704 is formed by changing the direction of liquid feeding. As shown in (h-1) of FIG. 14H, the plug 801 does not come off when the liquid is flowing from the first path 403 to the second path 404. As shown in (h-2) of FIG. 14H, the plug 801 may be disengaged when the liquid flows from the second path 404 to the first path 403. In combination with the configuration of FIGS. 14G and 14A, the third path 704 may be reliably or stepwise formed by two triggers in the direction of heating and solution flow.

The plug 801 may be installed so as to be in contact with the outer periphery of the membrane 203, or may be installed inside the membrane 203. When the membrane 203 is fixed to the flow path substrate 409 with an adhesive, etc. as illustrated in FIG. 4B, the fixing material serves as the plug 801. When the plug 801 is detached or melted, the membrane 203 is detached, and the third path 704 can be formed. The third path 704 may be reliably formed by further applying pressure after the membrane 203 is detached.

FIG. 14I illustrates a configuration example in which the membrane 203 is fixed to the flow path substrate 409 by the ring 411, and the membrane 203 is provided with the plug 801. The membrane 203 is secured with a ring 411, and the plug 801 is removed to form a third path 704. In such a configuration, the membrane 203 is fixed to the flow path substrate 409 even after the third path 704 is formed. Therefore, when the electrophoresis reagent is introduced, the membrane 203 does not move, and the reagent can be introduced with good reproducibility. In addition, there is no concern that the moved membrane 203 blocks the flow path. On the other hand, if the membrane 203 is not fixed to the flow path substrate 409 from the beginning, or if the fixing is released at the time of forming the third path 704 and the membrane is removed at the time of reagent introduction, air bubbles may enter between the membrane 203 and the liquid, and the elution efficiency may vary. In addition, the membrane 203 may move to block the flow path. In the case of the form as shown in FIG. 14I, when air bubbles are caught on the membrane 203, the liquid is conveyed via the third path 704.

In other forms, a part or the whole of the membrane 203 may be made of a brittle material, and the third path 704 may be formed by removing or breaking the brittle material with blowing pressure, etc.

### Third Path Formation Timing

The third path 704 can be configured to be closed for a controlled timing and allow the solution to pass through at acontrolled timing. In particular, at the start of dissolution product transfer in step S607 of FIG. 6, the third path 704 can be configured to be closed. Since the third path 704 is closed, the nucleic acid contained in the dissolution product can be captured by the membrane 203 with high efficiency.

The third path 704 may be opened during the conveyance of dissolution product in step S607. Forensic samples often include highly viscous samples and samples mixed with dirt. When such dust is captured by the fine mesh membrane 203, clogging occurs and conveyance is stopped. Therefore, the clogging can be eliminated by forming the third path 704 when a certain time has lapsed at a certain pressure or a certain temperature during the conveyance. The third path 704 may be formed so that the cross-sectional area gradually increases.

The third path 704 may be opened immediately after completion of step S607 and before step S608. When the dissolution product completely escapes from the membrane 203 and is conveyed to the waste liquid chamber 205, gas or air bubbles in the subsequent stage may be caught by the membrane 203, and a gas-liquid interface may be generated. The pressure required to fully vent the gas-liquid interface is higher than the pressure required to transport the dissolution product. By forming the third path 704, the generation of pressure necessary for the gas-liquid interface to pass through the membrane 203 can be avoided. In particular, even if the liquid (washing liquid, etc.) conveyed after the dissolution product is not necessarily conveyed so as to pass through the inside of the membrane 203, the effect can be sufficiently exhibited as long as the liquid is conveyed so as to be partially in contact with the membrane 203. Therefore, even if the third path 704 is opened after completion of conveyance of the dissolution product, the sensitivity is not lowered.

In step S608, the third path 704 may be opened during washing. If the washing liquid is ethanol, a method of plugging the third path 704 with a material that dissolves in ethanol may be taken. If the material completely dissolves in the first half of the washing, it does not remain in the amplification in the subsequent stage. If the third path 704 is formed before or in the middle of washing, the differential pressure required for transporting the washing liquid can be reduced, and the transport time can be reduced.

By forming the third path 704 after step S608, the efficiency of capture and washing can be maintained high.

In step S609, a third path may be formed when drying the membrane 203. The pressure difference during drying may be utilized to release the third path. Since the third path 704 is formed at the time of drying, even a pump having a small output can gain a flow rate, so that drying can be performed more quickly and reliably.

As described above, in order to achieve both highly efficient capture of a biomolecule from a first solution (dissolution product containing a biomolecule) on a membrane and rapid progress of conveyance of a fluid performed at a subsequent stage, such as conveyance of a dissolution product and a washing liquid conveyance/drying step, it is effective that the flow path device includes a space in which a liquid is stored before and after the membrane, and a first path and a second path connected to the space, and at the start of capture of a biomolecule, a first space close to the first path and a second space close to the second path in the space are separated by the membrane, and a third path in which a fluid can move between the first space and the second space is newly formed until the entire processing step is ended.

By opening the third path 704 before step S610, it is possible to prevent air bubbles from biting the membrane 203 at the time of introduction of the amplification reagent and to prevent the elution efficiency from being lowered. By appropriately stirring, the reagent can be brought into contact with the membrane 203 even if air bubbles are caught. In addition, the flow path resistance at the time of introducing the amplification reagent is low, and the reproducibility is high. Since the amplification reagent can be introduced without passing through the membrane 203, the capture of the amplification component by the membrane 203 can be minimized.

When the third path 704 is released by heating, the amplification reagent may be introduced while heating in step S610 to form the third path 704. The third path 704 may also be formed during amplification.

If the third path 704 is formed before the amplified product is transported by the electrophoresis reagent in step S613, the transportation of the amplified product or the electrophoresis sample can be easily completed.

The third path 704 may gradually spread in each step. When all the third paths 704 are formed at a time, the flow path resistance greatly fluctuates, and there is a possibility that the liquid splashes. In addition, it is possible to avoid the risk of clogging the flow path with the plug 801 by gradually releasing the third path 704 that has been filled.

### Summary

As described above, the analysis system 1 (biomolecule processing apparatus) of the present disclosure includes the flow path device 200 that captures and amplifies the nucleic acid (biomolecule) in the sample (sample) to be introduced, and the computer 100 (controlling device) that controls the conveyance of the fluid in the analysis system 1.

The flow path device 200 includes a membrane 203 that captures nucleic acid contained in a sample, a nucleic acid capturing/amplification chamber 202 having a space in which liquid is accumulated before and after the membrane 203, and a first path 403 and a second path 404 connected to the space. The computer 100 performs a process of capturing nucleic acid on the membrane 203 by conveying a liquid containing nucleic acid from the first path 403 to the second path 404, and a process of amplifying nucleic acid together with the membrane 203 using the membrane 203 and the liquid in the space after the capturing. During the capturing, the first space 401 close to the first path 403 and the second space 402 close to the second path 404 are separated by the membrane 203, and the third path 704 in which the fluid can move between the first space 401 and the second space 402 is newly formed until the amplification reaction is ended after the capturing is ended.

The flow path device 200 of the present disclosure has a simple flow path structure. Then, the nucleic acid can be captured with high efficiency in the nucleic acid capturing/amplification chamber 202, and the solution can be stably conveyed by forming the third path 704. According to the analysis system 1 including the flow path device 200, the sample can be analyzed with high sensitivity and in a short time.

### Modified Example

The present disclosure is not limited to the above-described embodiments, and includes various modifications. For example, the above-described embodiments have been described in detail in order to describe the present disclosure in an easy-to-understand manner, and do not necessarily have all the described configurations. Further, a part of one embodiment can be replaced with a configuration of another embodiment. In addition, the configuration of another embodiment can be added to the configuration of a certain embodiment. In addition, for a part of the configuration of each embodiment, a part of the configuration of another embodiment can be added, deleted, or replaced.

### Reference Signs List

1 analysis system

100 computer (controlling device)

200 flow path device

202 nucleic acid capturing/amplification chamber (space)

203 membrane

401 first space

402 second space

403 first path

404 second path

409 flow path substrate

701 amplification reagent

703 electrophoresis reagent

704 third path

300 detection unit

B air bubble

1. A method for processing a biomolecule using a flow path device configured to capture a biomolecule in a sample to be introduced, the flow path device comprising:
a membrane configured to capture the biomolecule contained in the sample;
a space in which a liquid is accumulated before and after the membrane; and
a first path and a second path connected to the space, the method for processing a biomolecule comprising
conveying a first liquid containing the biomolecule from the first path to the second path, and capturing the biomolecule on the membrane, wherein
at the start of the capturing, a first space close to the first path and a second space close to the second path in the space are separated by the membrane, and
the method further comprising newly forming a third path through which a fluid can move between the first space and the second space.

2. The method for processing a biomolecule according to claim 1, further comprising bringing a second liquid into contact with the membrane via the first path or the second path after the conveying of the first liquid.

3. The method for processing a biomolecule according to claim 2, wherein the biomolecule is a nucleic acid.

4. The method for processing a biomolecule according to claim 3, further comprising:
amplifying the nucleic acid together with the membrane after the nucleic acid is captured by the membrane, wherein
the second liquid is a liquid containing a reagent necessary for the amplification of the nucleic acid, and
the third path is formed between the end of the capturing and the end of the amplification.

5. The method for processing a biomolecule according to claim 1, wherein a flow path resistance of the third path is smaller than a flow path resistance of the membrane.

6. The method for processing a biomolecule according to claim 2, further comprising transferring the second liquid from the first space or the second space to the second space or the first space via the third path.

7. The method for processing a biomolecule according to claim 4, further comprising at least one of:
collecting an amplified product of the biomolecule from the second path or the first path by pressurizing the first path or the second path;
or
conveying the amplified product by depressurizing the first path or the second path to draw in a fluid of the second path or the first path.

8. The method for processing a biomolecule according to claim 1, wherein the forming the third path comprises heating the space to 50°C or higher.

9. The method for processing a biomolecule according to claim 1, wherein the third path is filled with a material that is deformed by heat during the capturing, and
forming the third path comprises transferring the material that is deformed by heat into the first space or the second space after the capturing is ended.

10. The method for processing a biomolecule according to claim 1, wherein the forming the third path comprises adjusting a differential pressure between the first space and second space to 50 kPa or higher.

11. The method for processing a biomolecule according to claim 2, wherein forming the third path is performed before introducing the second liquid into the space.

12. The method for processing a biomolecule according to claim 2, wherein the second liquid is introduced from the first path and transfers to the second space via the third path.

13. The method for processing a biomolecule according to claim 3, wherein the membrane contains silica or cellulose as a main component.

14. The method for processing a biomolecule according to claim 4, wherein when the biomolecule is captured on the membrane, the biomolecule is dissolved in a solution having a higher salt concentration or a lower pH than that of the amplification reagent.

15. A device for processing a biomolecule, comprising:
a flow path device in which capturing and amplification of a biomolecule in a sample to be introduced are performed; and
a controlling device configured to control conveying of a fluid in the biomolecule processing device,
the flow path device comprising:
a membrane configured to capture the biomolecule contained in the sample;
a space in which a liquid is accumulated before and after the membrane; and
a first path and a second path connected to the space, the controlling device performing
a process of conveying a first liquid containing the biomolecule from the first path to the second path, and capturing the biomolecule on the membrane; and
a process in which the biomolecule is amplified together with the membrane by using the membrane and the liquid in the wherein
during the capturing, a first space close to the first path and a second in the space, space close to the second path in the space are separated by the membrane, and
a third path through which a fluid can move between the first space and the second space is newly formed from the end of the capturing to the end of the amplification.
